(19) United States
(12) Patent Application Publication
(10) Pub. No.: US 2011/0009560 A1
(43) Pub. Date: Jan. 13, 2011

(54) ETHYLENICALLY UNSATURATED MONOMERS COMPRISING ALIPHATIC AND AROMATIC MOIETIES

(75) Inventors: Robert E. Hefner, JR., Rosharon, TX (US); Michael J. Mullins, Houston, TX (US); Mark B. Wilson, Clute, TX (US); Ulrich Herold, Buehl (DE)

Correspondence Address: The Dow Chemical Company, P.O. BOX 1967, 2040 Dow Center, Midland, MI, 48641, US

(73) Assignee: Dow Global Technologies Inc., Midland, MI (US)

(21) Appl. No.: 12/921,846

(22) PCT Filed: Mar. 9, 2009
(86) PCT No.: PCT/US2009/036522

§ 371 Date: Sep. 10, 2010

Related U.S. Application Data
(60) Provisional application No. 61/035,823, filed on Mar. 12, 2008.

(51) Int. Cl. C08L 75/04; C08G 18/10; C08G 65/38; C07C 43/205

(52) U.S. Cl. 524/590; 528/60; 528/219; 568/640

(57) ABSTRACT

Polymerizable monomers comprising at least one 1- or 2-propylene moiety and further comprising both aromatic moieties and additional aliphatic moieties and polymerizable mixtures, resins and thermoset products based on these monomers.

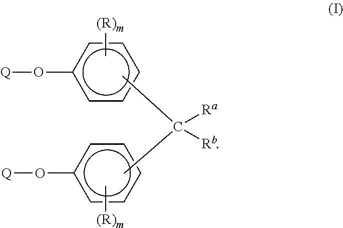

### BACKGROUND OF THE INVENTION

1. Field of the Invention

The present invention relates generally to polymerizable monomers which comprise at least one 1- or 2-propylene moiety and further comprise both aromatic moieties and additional aliphatic moieties and to resins and thermoset products based on these monomers.

2. Discussion of Background Information

The performance requirements for thermosetting resins used in electrical applications continue to escalate. In particular, high frequency electronics have become more commonplace with advances in computer, communications, and wireless technologies. In view thereof, there is a need for resins which show reduced dielectric constants and dissipation factors as well as enhanced thermal resistance.

Aromatic cyanate esters have been used in electronics applications for many years. The most common cyanate ester, bisphenol A dicyanate, is prepared by reaction of bisphenol A (isopropylidene diphenol) with a cyanogen halide, for example, cyanogen bromide, in the presence of an acid acceptor, for example, triethylamine. One route to thermoset resins with desired property improvements has involved development of copolymerizable mixtures of aromatic cyanate esters and one or more other monomers. Most commonly encountered are the copolymers of aromatic cyanate esters and bis(maleimides). Also known are copolymers of aromatic cyanate esters (or aromatic cyanamides) with ethylenically unsaturated polymerizable monomers including allyl monomers, with diallyl bisphenol A being most notable.

### SUMMARY OF THE INVENTION

It is believed that the dielectric properties and thermal resistance of thermosets produced from di- and polycyanates can be improved by increasing the hydrocarbon content of the thermoset matrix. One such method is to increase the hydrocarbon content of the di- or polycyanate monomer used. The present inventors have now found another method for increasing the hydrocarbon content of the thermoset matrix, i.e., through the use of hydrocarbon-rich polymerizable ethylenically unsaturated monomers which can be copolymerized with, e.g., cyanate monomers.

Specifically, the present inventors have found, inter alia, a class of monomers which contain a high percentage of non-polar hydrocarbon groups. While the art might have predicted that the incorporation of a hydrocarbon structure would be deleterious to the thermal properties and the cure profile of a thermosettable mixture incorporating these monomers, the exact opposite was observed (see Examples and Comparative Experiments below). Thus, the hydrocarbon portion of the monomers was found to be desirable because it affords enhanced thermal resistance, low moisture absorption, and excellent dielectric properties, without a deleterious effect on the cure behavior of a thermosettable mixture prepared therefrom. It was unexpectedly found that the increased hydrocarbon content of the monomers of the present invention can moderate the enthalpic cure energy without increasing the cure onset and end temperatures. This reduction in exothermicity on cure can help to prevent damage such as cracking or delamination which may result from the cure of monomers which comprise a smaller proportion of non-polar hydrocarbon groups than the monomers of the present invention.

The present invention provides ethylenically unsaturated monomers of formula (I):

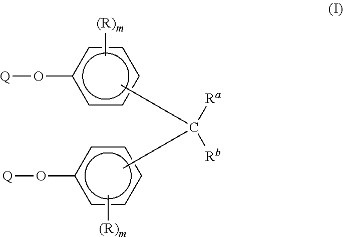

wherein:
each m independently is 0, 1, or 2;
the moieties R^{a} and R^{b} independently represent optionally substituted aliphatic groups comprising a total of from about 5 to about 24 carbon atoms, and R^{a} and R^{b} together with the carbon atom to which they are bonded may form an optionally substituted and/or optionally unsaturated and/or optionally polycyclic aliphatic ring structure; and
the moieties R independently represent halogen, cyano, nitro, hydroxy, amino optionally carrying one or two alkyl groups, optionally substituted alkyl, optionally substituted cycloalkyl, optionally substituted alkoxy, optionally substituted alkenyl, optionally substituted alkenyloxy, optionally substituted aryl, optionally substituted aralkyl, optionally substituted aryloxy, and optionally substituted aralkoxy; and the moieties Q independently represent hydrogen, HR¹C═CR¹—CH₂— or H₂R¹C—CR¹═HC—, wherein the moieties R¹ independently represent hydrogen or optionally substituted alkyl having from 1 to about 3 carbon atoms;
with the proviso that when both moieties Q are hydrogen and R^{a} and R^{b} together with the carbon atom to which they are bonded do not form an aliphatic ring structure having at least about 8 (e.g., at least about 9 or at least about 10) ring members, at least one moiety R represents HR¹C═CR¹—CH₂— or H₂R¹C—CR¹═HC—.

In one aspect, the monomers of formula (I) may be ethylenically unsaturated monomers of formula (Ia):

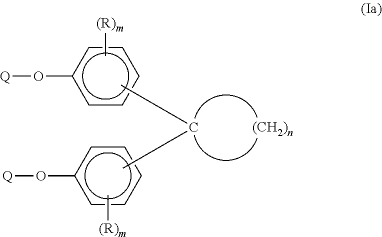

wherein:
n has a value of from about 5 to about 24;
each m independently is 0, 1, or 2;
the moieties R independently represent halogen, cyano (—CN), nitro, hydroxy, amino optionally carrying one or two alkyl groups preferably having from 1 to about 6 carbon atoms, unsubstituted or substituted alkyl preferably having from 1 to about 6 carbon atoms, unsubstituted or substituted cycloalkyl preferably having from about 5 to about 8 carbon atoms, unsubstituted or substituted alkoxy preferably having from 1 to about 6 carbon atoms, unsubstituted or substituted alkenyl preferably having from 3 to about 6 carbon atoms, unsubstituted or substituted alkenyloxy preferably having from 3 to about 6 carbon atoms, unsubstituted or substituted aryl preferably having from 6 to about 10 carbon atoms, unsubstituted or substituted aralkyl preferably having from 7 to about 12 carbon atoms, unsubstituted or substituted aryloxy preferably having from 6 to about 10 carbon atoms, and unsubstituted or substituted aralkoxy preferably having from 7 to about 12 carbon atoms; and
the moieties Q independently represent hydrogen, HR¹C═CR¹—CH₂—, or H₂R¹C—CR¹═HC—, wherein the moieties R¹ independently represent hydrogen or unsubstituted or substituted alkyl having from 1 to about 3 carbon atoms, with the proviso that when both moieties Q are hydrogen, at least one moiety R represents HR¹C═CR¹—CH₂— or H₂R¹C—CR¹═HC—;
and any non-aromatic cyclic moieties comprised in the above formula (Ia) may optionally carry one or more substituents and/or may optionally comprise one or more double bonds and/or may optionally be polycyclic.

In one aspect of the monomers of formula (Ia), n may have a value of from about 9 to about 16; for example, n may have a value of 9, 10, or 11 and may in particular equal 11.

In another aspect of the monomers of formula (I)/(Ia), each m may independently be 0 or 1.

In yet another aspect of the monomers of formula (I)/(Ia), the moieties Q may independently represent HR¹C═CR¹—CH₂— or H₂R¹C—CR¹═HC—.

In a still further aspect, the moieties R¹ may independently represent hydrogen or methyl. For example, the moieties Q may be identical and may represent allyl (=2-propenyl), methallyl (=2-methyl-2-propenyl), or 1-propenyl.

Non-limiting examples of the monomers of formula (I) include 1,1-bis(4-hydroxyphenyl)cyclododecane bis(allyl ether), 1,1-bis(4-hydroxyphenyl)-cyclododecane bis(methallyl ether), 1,1-bis(4-hydroxyphenyl)cyclodecane bis(allyl ether), 1,1-bis(4-hydroxyphenyl)cyclodecane bis(methallyl ether), 2,2-bis(4-hydroxyphenyl)adamantane bis(allyl ether), 2,2-bis(4-hydroxyphenyl)adamantane bis(methallyl ether), 4,4′-bis(4-hydroxyphenyl)octahydro-1,4:5,8-dimethanonaphthalen-2(1H)ylidene bis(allyl ether), 4,4′-bis(4-hydroxyphenyl)-octahydro-1,4:5,8-dimethanonaphthalen-2(1H)ylidene bis(methallyl ether), 5,5-bis(4-hydroxyphenyl)hexahydro-4,7-methanoindane bis(allyl ether) and 5,5-bis(4-hydroxyphenyl)hexahydro-4,7-methanoindane bis(methallyl ether), partial or complete Claisen rearrangement products of 1,1-bis(4-hydroxyphenyl)cyclododecane bis(allyl ether), and monomers which carry at least one ortho-substituent on at least one aromatic ring to block a Claisen rearrangement such as, e.g., 1,1-bis(4-hydroxy-3,5-dimethylphenyl)cyclododecane bis(allyl ether), 1,1-bis(4-hydroxy-3,5-dimethylphenyl)cyclododecane bis(methallyl ether), 1,1-bis(4-hydroxy-3-methylphenyl)cyclododecane bis(allyl ether) and 1,1-bis(4-hydroxy-3-methylphenyl)cyclododecane bis(methallyl ether). A preferred example of the monomers of formula (I) is 1,1-bis(4-hydroxyphenyl)cyclododecane bis(allyl ether)=1,1-bis[4-(2-propenyloxy)phenyl]cyclododecane.

The present invention also provides ethylenically unsaturated monomers of formula (II):

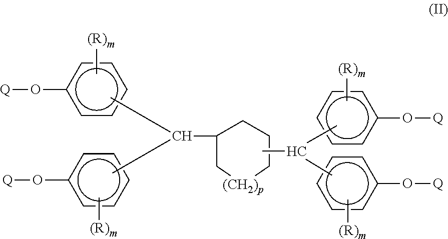

wherein:
p is 0 or an integer of from 1 to about 19;
each m independently is 0, 1, or 2;
the moieties R independently represent halogen, cyano, nitro, hydroxy, amino optionally carrying one or two alkyl groups having from 1 to about 6 carbon atoms, unsubstituted or substituted alkyl preferably having from 1 to about 6 carbon atoms, unsubstituted or substituted cycloalkyl preferably having from about 5 to about 8 carbon atoms, unsubstituted or substituted alkoxy preferably having from 1 to about 6 carbon atoms, unsubstituted or substituted alkenyl preferably having from 3 to about 6 carbon atoms, unsubstituted or substituted alkenyloxy preferably having from 3 to about 6 carbon atoms, unsubstituted or substituted aryl preferably having from 6 to about 10 carbon atoms, unsubstituted or substituted aralkyl preferably having from 7 to about 12 carbon atoms, unsubstituted or substituted aryloxy preferably having from 6 to about 10 carbon atoms, and unsubstituted or substituted aralkoxy preferably having from 7 to about 12 carbon atoms; and
the moieties Q independently represent hydrogen, HR¹C═CR¹—CH₂—, or H₂R¹C—CR¹═HC—, wherein the moieties R¹ independently represent hydrogen or unsubstituted or substituted alkyl having from 1 to about 3 carbon atoms, with the proviso that when all four moieties Q are hydrogen, at least one moiety R represents HR¹C═CR¹—CH₂— or H₂R¹C—CR¹═HC—;
and any non-aromatic cyclic moieties comprised in the above formula (II) may optionally carry one or more substituents and/or may optionally comprise one or more double bonds.

In one aspect of the monomers of the above formula (II), p may have a value of from 1 to about 14. For example, p may have a value of 1, 2, or 3 and may in particular equal 1.

In another aspect of the monomers of formula (II), each m may independently be 0 or 1.

In yet another aspect, the moieties Q may independently represent HR¹C═CR¹—CH₂— or H₂R¹C—CR¹═HC—.

In a still further aspect, the moieties R¹ may independently represent hydrogen or methyl. For example, the moieties Q may be identical and represent allyl (=2-propenyl), methallyl (=2-methyl-2-propenyl), or 1-propenyl.

Non-limiting examples of the monomers of the above formula (II) include dimethylcyclohexane tetraphenol tetra(allyl ether), dimethylcyclohexane tetraphenol tetra(methallyl ether), dimethylcyclohexane tetraphenol tetra(1-propenyl ether), dimethylcyclooctane tetraphenol tetra(allyl ether), dimethylcyclooctane tetraphenol tetra(methallyl ether), dimethylcyclooctane tetraphenol tetra(1-propenyl ether), partial or complete Claisen rearrangement products of dimethylcyclohexane tetraphenol tetra(allyl ether), and monomers which carry at least one substituent on at least one aromatic ring to block a Claisen rearrangement. A preferred example of the monomers of formula (II) is dimethylcyclohexane tetraphenol tetra(allyl ether).

The present invention also provides polymers (i.e., homo- and copolymers) and prepolymers of the ethylenically unsaturated monomers of formulae (I)/(Ia) and (II) set forth above (including the various aspects thereof).

The present invention also provides a first polymerizable mixture which comprises at least two of (i) at least one monomer of the above formula (I)/(Ia) and/or a prepolymer thereof, (ii) at least one monomer of the above formula (II) and/or a prepolymer thereof, and (iii) at least one monomer and/or a prepolymer thereof which is different from monomers of the above formulae (I)/(Ia) and (II).

In one aspect of the first mixture, the at least one monomer (iii) may be selected from monomers which comprise one or more polymerizable ethylenically unsaturated moieties, aromatic di- and polycyanates, aromatic di- and polycyanamides, di- and polymaleimides, and di- and polyglycidyl ethers.

In another aspect, the first mixture may comprise at least components (i) and (iii), or it may comprise at least components (ii) and (iii).

In another aspect, component (iii) of the first mixture may comprise a dicyanate compound of the following formula (III) and/or a prepolymer thereof:

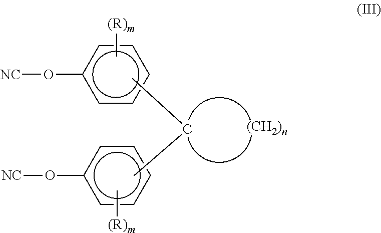

wherein:
n has a value of from about 5 to about 24;
each m independently is 0, 1, or 2;
the moieties R independently represent halogen, cyano, nitro, unsubstituted or substituted alkyl preferably having from 1 to about 6 carbon atoms, unsubstituted or substituted cycloalkyl preferably having from about 5 to about 8 carbon atoms, unsubstituted or substituted alkoxy preferably having from 1 to about 6 carbon atoms, unsubstituted or substituted alkenyl preferably having from 3 to about 6 carbon atoms, unsubstituted or substituted alkenyloxy preferably having from 3 to about 6 carbon atoms, unsubstituted or substituted aryl preferably having from 6 to about 10 carbon atoms, unsubstituted or substituted aralkyl preferably having from 7 to about 12 carbon atoms, unsubstituted or substituted aryloxy preferably having from 6 to about 10 carbon atoms, and unsubstituted or substituted aralkoxy preferably having from 7 to about 12 carbon atoms;
and any non-aromatic cyclic moieties comprised in the above formula (III) may optionally carry one or more substituents and/or may optionally comprise one or more double bonds and/or may optionally be polycyclic.

In one aspect of the above dicyanate compound, n may have a value of from about 9 to about 16. For example, n may have a value of 9, 10, or 11 and may in particular equal 11. In another aspect, each m may independently be 0 or 1. A specific (and preferred) example of a dicyanate compound of formula (III) is 1,1-bis(4-cyanatophenyl)cyclododecane.

In another aspect of the first mixture, component (iii) thereof may comprise a polycyanate compound of formula (IV) and/or a prepolymer thereof:

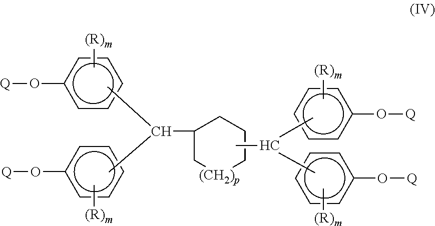

wherein:
p is 0 or an integer of from 1 to about 19;
each m independently is 0, 1, or 2;
the moieties R independently represent halogen, cyano, nitro, unsubstituted or substituted alkyl preferably having from 1 to about 6 carbon atoms, unsubstituted or substituted cycloalkyl preferably having from about 5 to about 8 carbon atoms, unsubstituted or substituted alkoxy preferably having from 1 to about 6 carbon atoms, unsubstituted or substituted alkenyl preferably having from 3 to about 6 carbon atoms, unsubstituted or substituted alkenyloxy preferably having from 3 to about 6 carbon atoms, unsubstituted or substituted aryl preferably having from 6 to about 10 carbon atoms, unsubstituted or substituted aralkyl preferably having from 7 to about 12 carbon atoms, unsubstituted or substituted aryloxy preferably having from 6 to about 10 carbon atoms, and unsubstituted or substituted aralkoxy preferably having from 7 to about 12 carbon atoms; and
at least two of the moieties Q represent —CN and the remaining moieties Q represent hydrogen;
and any non-aromatic cyclic moieties comprised in the above formula (IV) may optionally carry one or more substituents and/or may optionally comprise one or more double bonds.

In one aspect of the above polycyanate compound, all four moieties Q may represent —CN. In another aspect, each m may independently be 0 or 1 and/or p may have a value of from 1 to about 14. For example, p may have a value of 1, 2, or 3 and may in particular equal 1. A specific example of a polycyanate compound of formula (IV) is dimethylcyclohexane tetraphenol tetracyanate.

In another aspect of the first mixture, the mixture may further comprise one or more substances which are selected from polymerization catalysts, co-curing agents, flame retardants, synergists for flame retardants, solvents, fillers, adhesion promoters, wetting aids, dispersing aids, surface modifiers, thermoplastic polymers, and mold release agents.

The present invention also provides a second mixture which comprises at least one ethylenically unsaturated monomer of the above formula (I)/(Ia) and/or a prepolymer thereof and one or more substances which are selected from polymerization catalysts, co-curing agents, flame retardants, synergists for flame retardants, solvents, fillers, adhesion promoters, wetting aids, dispersing aids, surface modifiers, thermoplastic polymers, and mold release agents. For example, the second mixture may be substantially free of polymerizable monomers and/or monomers which are copolymerizable with the at least one ethylenically unsaturated monomer of the above formula (I)/(Ia).

The present invention also provides a third mixture which comprises at least one ethylenically unsaturated monomer of the above formula (II) and/or a prepolymer thereof and one or more substances which are selected from polymerization catalysts, co-curing agents, flame retardants, synergists for flame retardants, solvents, fillers, glass fibers, adhesion promoters, wetting aids, dispersing aids, surface modifiers, thermoplastic polymers, and mold release agents.

In one aspect, each of the first, second and third mixtures set forth above (including the various aspects thereof) may be partially polymerized (e.g., prepolymerized or B-staged) or completely polymerized and the present invention also provides a product which comprises such a partially or completely polymerized (preferably substantially completely polymerized) mixture. For example, the product or part thereof may be an electrical laminate, an IC (integrated circuit) substrate, a casting, a coating, a die attach and mold compound formulation, a composite, and an adhesive.

The present invention also provides a process for preparing a mixture of ethylenically unsaturated monomers, which mixture may, for example, comprise one or more of the ethylenically unsaturated monomers of the above formula (II). The process comprises the condensation of a dialdehyde of a cycloalkane having from about 5 to about 24 ring carbon atoms with a hydroxyaromatic (e.g., phenolic) compound at a ratio of aromatic hydroxy groups to aldehyde groups which affords a mixture of polyphenolic compounds with a polydispersity of not higher than about 2, e.g., not higher than about 1.8, not higher than about 1.5, or not higher than about 1.3. The mixture of polyphenolic compounds may then be subjected to an etherification reaction to partially or completely convert the aromatic hydroxy groups which are present in the mixture into ether groups of formula HR¹C═CR¹—CH₂—O— and/or H₂R¹C—CR¹═HC—O—, wherein the moieties R¹ independently represent hydrogen or unsubstituted or substituted alkyl having from 1 to about 3 carbon atoms.

In one aspect of the process, the ratio of the number of aromatic hydroxy groups to the number of aldehyde groups may be at least about 4, e.g., at least about 5, at least about 5.5, or at least about 6.

In another aspect of the process, the cycloalkane may have from about 6 to about 19 ring carbon atoms, for example, 6, 7, or 8 ring carbon atoms and in particular 6 ring carbon atoms.

In another aspect, the dialdehyde may comprise a cyclohexane dicarboxaldehyde (e.g., 1,3-cyclohexane dicarboxaldehyde and/or 1,4-cyclohexane dicarboxaldehyde) and/or the hydroxyaromatic compound may comprise phenol.

In yet another aspect of the method, the moieties R¹ may independently represent hydrogen or methyl. For example, the groups of formula HR¹C═CR¹—CH₂—O— and/or H₂R¹C—CR¹═HC—O— may represent allyl, methallyl, or 1-propenyl.

The present invention also provides a mixture of ethylenically unsaturated monomers which are obtainable by the process set forth above (including the various aspects thereof), either as such, or in partially polymerized (e.g., prepolymerized or B-staged) or completely polymerized and/or partially or completely copolymerized form.

In one aspect of this mixture, the polydispersity of the mixture may be not higher than about 1.8, e.g., not higher than about 1.5, or not higher than about 1.3, and/or the average number of hydroxy groups per molecule may be at least about 4, e.g., at least about 5, or at least about 6.

Other features and advantages of the present invention will be set forth in the description of invention that follows, and will be apparent, in part, from the description or may be learned by practice of the invention. The invention will be realized and attained by the compositions, products, and methods particularly pointed out in the written description and claims hereof.

### DETAILED DESCRIPTION OF THE PRESENT INVENTION

Unless otherwise stated, a reference to a compound or component includes the compound or component by itself, as well as in combination with other compounds or components, such as mixtures of compounds.

As used herein, the singular forms “a,” “an,” and “the” include the plural reference unless the context clearly dictates otherwise.

Except where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not to be considered as an attempt to limit the application of the doctrine of equivalents to the scope of the claims, each numerical parameter should be construed in light of the number of significant digits and ordinary rounding conventions.

Additionally, the recitation of numerical ranges within this specification is considered to be a disclosure of all numerical values and ranges within that range. For example, if a range is from about 1 to about 50, it is deemed to include, for example, 1, 7, 34, 46.1, 23.7, or any other value or range within the range.

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show embodiments of the present invention in more detail than is necessary for the fundamental understanding of the present invention, the description making apparent to those skilled in the art how the several forms of the present invention may be embodied in practice.

As set forth above, the present invention provides, inter alfa, ethylenically unsaturated monomers of formula (I):

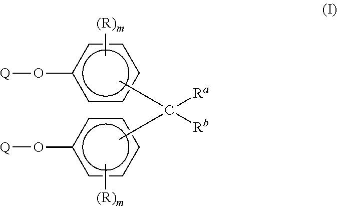

The moieties R^{a} and R^{b} in the above formula (I) may independently represent optionally substituted aliphatic groups comprising a total of from about 5 to about 24 carbon atoms. Usually, the total number of carbon atoms in the aliphatic moieties R^{a} and R^{b} will be at least about 6, e.g., at least about 7, at least about 8, at least about 9, or at least about 10, but will usually be not higher than about 18, e.g., not higher than about 16, or not higher than about 12. The aliphatic moieties may be linear, branched or cyclic and saturated or unsaturated. Non-limiting examples thereof are linear or branched alkyl groups and alkenyl groups, cycloalkyl and cycloalkenyl groups as well as alkylcycloalkyl and cycloalkylalkyl groups such as, e.g., methyl, ethyl, n-propyl, isopropyl, n-butyl, n-pentyl, n-hexyl, n-heptyl, n-octyl, n-nonyl, n-decyl, n-undecyl, n-dodecyl, cyclohexyl, methylcyclohexyl, and cyclohexylmethyl, and the corresponding mono- and diunsaturated groups. Further, these groups may be substituted by one or more (e.g., 1, 2, 3, or 4) substituents. Non-limiting examples of substituents are F, Cl and Br, as well as aromatic groups (such as, e.g., phenyl). Also, often one of the moieties R^{a} and R^{b} will represent methyl or ethyl, in particular, methyl.

The moieties R^{a} and R^{b} in the above formula (I) may also form, together with the carbon atom to which they are bonded, an optionally unsaturated and/or optionally substituted and/or optionally polycyclic aliphatic ring structure which has at least about 6 ring carbon atoms. Examples of corresponding compounds are those of formula (Ia):

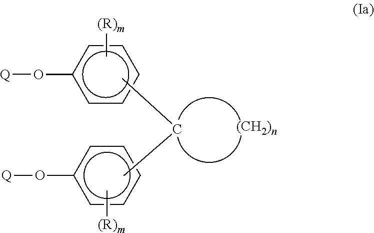

The value of n in the above formula (Ia) is not lower than about 5, e.g., not lower than about 6, not lower than about 7, not lower than about 8, not lower than about 9, or not lower than about 10, and not higher than about 24, e.g., not higher than about 16, not higher than about 14, or not higher than about 12, and preferably equals 8, 9, 10, 11, or 12, in particular 11 (i.e., giving rise to a cyclododecylidene structure).

The cycloaliphatic moiety shown in the above formula (Ia) may optionally comprise one or more (e.g., 1, 2, 3, or 4) double bonds and/or may carry one or more (e.g., 1, 2, or 3) substituents and/or may optionally be polycyclic (e.g., bicyclic or tricyclic). If more than one substituent is present, the substituents may be the same or different. Non-limiting examples of substituents which may be present on the cycloaliphatic moiety are alkyl groups, e.g., optionally substituted alkyl groups having from 1 to about 6 carbon atoms (e.g., methyl or ethyl), hydroxy, amino which optionally carries one or two alkyl groups preferably having from 1 to about 6 carbon atoms and halogen atoms such as, e.g., F, Cl, and Br. The alkyl groups may be substituted with, for example, one or more halogen atoms such as, e.g., F, Cl, and Br.

The value of each m in the above formula (I)/(Ia) independently is 0, 1, or 2. Preferably, the values of m are identical and/or are 0 or 1.

The moieties R in the above formula (I)/(Ia) independently represent halogen (e.g., F, Cl, and Br, preferably Cl or Br), cyano, nitro, hydroxy, amino optionally carrying one or two alkyl groups preferably having from 1 to about 6 carbon atoms, unsubstituted or substituted alkyl preferably having from 1 to about 6 carbon atoms, unsubstituted or substituted cycloalkyl preferably having from about 5 to about 8 carbon atoms, unsubstituted or substituted alkoxy preferably having from 1 to about 6 carbon atoms, unsubstituted or substituted alkenyl preferably having from 3 to about 6 carbon atoms, unsubstituted or substituted alkenyloxy preferably having from 3 to about 6 carbon atoms, unsubstituted or substituted aryl preferably having from 6 to about 10 carbon atoms, unsubstituted or substituted aralkyl preferably having from 7 to about 12 carbon atoms, unsubstituted or substituted aryloxy preferably having from 6 to about 10 carbon atoms, and unsubstituted or substituted aralkoxy preferably having from 7 to about 12 carbon atoms.

It is to be appreciated that whenever the terms “alkyl” and “alkenyl” are used in the present specification and the appended claims, these terms also include the corresponding cycloaliphatic groups such as, e.g., cyclopentyl, cyclohexyl, cyclopentenyl, and cyclohexenyl. Also, where two alkyl and/or alkenyl groups are attached to two (preferably adjacent) carbon atoms of an aliphatic or aromatic ring, they may be combined to form an alkylene or alkenylene group which together with the carbon atoms to which this group is attached results in a preferably 5- or 6-membered ring structure. In the case of non-adjacent carbon atoms, this ring structure may give rise to a bicyclic compound.

The above alkyl groups R (including the alkyl groups which may be present in the above amino group which may carry one or two alkyl groups) and alkoxy groups will often comprise from 1 to about 4 carbon atoms and in particular, 1 or 2 carbon atoms. Non-limiting specific examples of these groups include, methyl, ethyl, propyl, isopropyl, n-butyl, isobutyl, sec-butyl, tert-butyl, methoxy, ethoxy, propoxy, isopropoxy, n-butoxy, isobutoxy, sec-butoxy, and tert-butoxy. The alkyl and alkoxy groups may be substituted with one or more (e.g., 1, 2, or 3) substituents. If more than one substituent is present, the substituents may be the same or different and are preferably identical. Non-limiting examples of these substituents include halogen atoms such as, e.g., F, Cl, and Br. Non-limiting specific examples of substituted alkyl and alkoxy groups include CF₃, CF₃CH₂, CCl₃, CCl₃CH₂, CHCl₂, CH₂Cl, CH₂Br, CCl₃O, CHCl₂O, CH₂ClO, and CH₂BrO.

The above alkenyl and alkenyloxy groups will often comprise 3 or 4 carbon atoms and in particular, 3 carbon atoms. Non-limiting specific examples of these groups are allyl, methallyl, and 1-propenyl. The alkenyl and alkenyloxy groups may be substituted with one or more (e.g., 1, 2, or 3) substituents. If more than one substituent is present, the substituents may be the same or different and are preferably identical. Non-limiting examples of these substituents include halogen atoms such as, e.g., F, Cl, and Br.

The above aryl and aryloxy groups will often be phenyl and phenoxy groups. The aryl and aryloxy groups may be substituted with one or more (e.g., 1, 2, 3, 4, or 5) substituents. If more than one substituent is present, the substituents may be the same or different. Non-limiting examples of these substituents include hydroxy, nitro, cyano, halogen such as, e.g., F, Cl, and Br, optionally halogen-substituted alkyl having from 1 to about 6 carbon atoms, e.g., from 1 to about 4 carbon atoms (for example, methyl or ethyl), optionally halogen-substituted alkoxy having from 1 to about 6 carbon atoms, e.g., from 1 to about 4 carbon atoms (for example, methoxy or ethoxy), and amino which may optionally carry one or more alkyl groups having from 1 to about 6 carbon atoms, e.g., from 1 to about 4 carbon atoms (for example, methyl or ethyl). Non-limiting specific examples of substituted aryl and aryloxy groups include, tolyl, xylyl, ethylphenyl, chlorophenyl, bromophenyl, tolyloxy, xylyloxy, ethylphenoxy, chlorophenoxy, and bromophenoxy.

The above aralkyl and aralkoxy groups will often be benzyl, phenethyl, benzyloxy, or phenethoxy groups. These groups may be substituted (preferably on the aryl ring, if at all) with one or more (e.g., 1, 2, 3, 4, or 5) substituents. If more than one substituent is present, the substituents may be the same or different. Non-limiting examples of these substituents include hydroxy, nitro, cyano, halogen such as, e.g., F, Cl, and Br, optionally halogen-substituted alkyl having from 1 to about 6 carbon atoms, e.g., from 1 to about 4 carbon atoms (for example, methyl or ethyl), optionally halogen-substituted alkoxy having from 1 to about 6 carbon atoms, e.g., from 1 to about 4 carbon atoms (for example, methoxy or ethoxy), and amino which may optionally carry one or more alkyl groups having from 1 to about 6 carbon atoms, e.g., from 1 to about 4 carbon atoms (for example, methyl or ethyl).

The moieties Q in the above formula (I)/(Ia) independently represent hydrogen, HR¹C═CR¹—CH₂— or H₂R¹C—CR¹═HC— wherein the moieties R¹ independently represent hydrogen or unsubstituted or substituted (preferably unsubstituted) alkyl having from 1 to about 3 carbon atoms. A preferred moiety Q is allyl. Further, it is preferred for the moieties Q to be identical. It also is preferred for the moieties Q to be different from hydrogen. Also preferably, at least one of the moieties Q is different from hydrogen.

Non-limiting specific examples of the above alkyl moieties R¹ include methyl, ethyl, propyl, and isopropyl. Methyl is preferred. If one or more substituents are present on these alkyl groups they may, for example, be halogen such as, e.g., F, Cl, and Br.

Non-limiting examples of the above monomers of formula (I)/(Ia) include 1,1-bis(4-hydroxyphenyl)cyclododecane bis(allyl ether), 1,1-bis(4-hydroxyphenyl)-cyclododecane bis(methallyl ether), 1,1-bis(4-hydroxyphenyl)-cyclododecane bis(1-propenyl ether), 1,1-bis(4-hydroxyphenyl)cyclodecane bis(allyl ether), 1,1-bis(4-hydroxyphenyl)cyclodecane bis(methallyl ether), 1,1-bis(4-hydroxyphenyl)-cyclodecane bis(1-propenyl ether), 2,2-bis(4-hydroxyphenyl)adamantane bis(allyl ether), 2,2-bis(4-hydroxyphenyl)adamantane bis(methallyl ether), 4,4′-bis(4-hydroxyphenyl)octahydro-1,4:5,8-dimethanonaphthalen-2(1H)ylidene bis(allyl ether), 4,4′-bis(4-hydroxyphenyl)octahydro-1,4:5,8-dimethanonaphthalen-2(1H)-ylidene bis(methallyl ether), 5,5-bis(4-hydroxyphenyl)hexahydro-4,7-methano-indane bis(allyl ether) and 5,5-bis(4-hydroxyphenyl)hexahydro-4,7-methanoindane bis(methallyl ether).

Further non-limiting examples of the above monomers of formula (I)/(Ia) include partial or complete Claisen rearrangement products of compounds of formula (I)/(Ia) wherein at least one of the moieties Q represents HR¹C═CR¹—CH₂— or H₂R¹C—CR¹═HC—. For example, in the case of the 1,1-bis(4-hydroxyphenyl)cyclododecane bis(allyl ether) such Claisen rearrangement products include compounds of formulae (A) and (B):

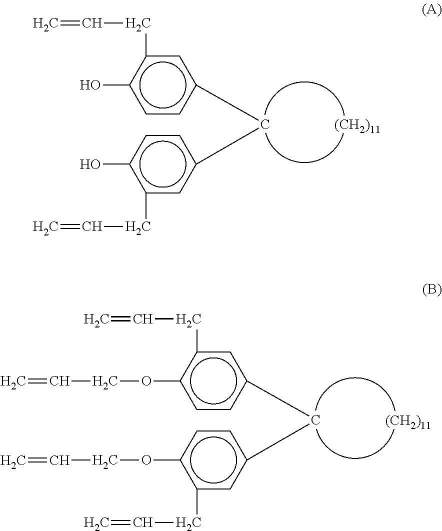

Further non-limiting examples of the above monomers of formula (I) include monomers which carry at least one substituent on at least one aromatic ring to block a Claisen rearrangement. A non-limiting specific example of such monomers is represented by formula (C):

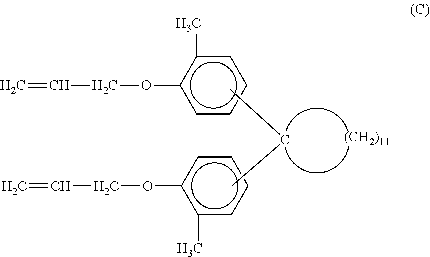

The monomers of formula (I)/(Ia) may prepared by methods which are well known to those of skill in the art. For example, these monomers may be prepared by etherification of a bisphenol of formula (V):

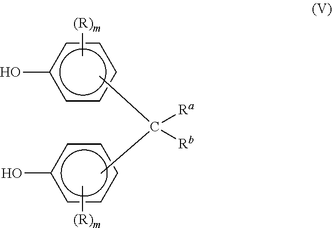

wherein m, R^{a}, R^{b} and R have the meanings set forth above with respect to formula (I) with a compound which comprises a group HR¹C═CR¹—CH₂— or H₂R¹C—CR¹═HC—.

The bisphenol of formula (V) can be prepared, for example, by condensation of phenols with ketones using methods well known in the art. Examples of these methods are described in, e.g., U.S. Pat. No. 4,438,241 and DE 3345945, the entire disclosures whereof are incorporated by reference herein. Generally speaking, the ketone is usually treated with a large excess of a phenol in the presence of an acid catalyst, non-limiting examples of which include mineral acids such as HCl or H₂SO₄, arylsulfonates, oxalic acid, formic acid, or acetic acid. A cocatalyst such as, e.g., a mercaptan may be added. Rather than using a soluble acid catalyst, it is also common to use a bed of sulfonated crosslinked polystyrene beads. Non-limiting examples of suitable ketone starting materials include cycloaliphatic ketones such as, e.g., cyclohexanone, 2-bromocyclohexanone, 2-chlorocyclohexanone, 2-methyl-cyclohexanone, 3-methylcyclohexanone, 4-methylcyclohexanone, 2-isopropylcyclohexanone, 3-isopropylcyclohexanone, 4-isopropylcyclohexanone, 2-n-butylcyclohexanone, 3-n-butylcyclohexanone, 4-n-butylcyclohexanone, 2-sec-butylcyclohexanone, 3-sec-butylcyclohexanone, 4-sec-butylcyclohexanone, 2-isobutylcyclohexanone, 3-isobutylcyclohexanone, 4-isobutylcyclohexanone, 2-t-butylcyclohexanone, 3-t-butylcyclohexanone, 4-t-butylcyclohexanone, 2,6-dimethylcyclohexanone, 2,4-diisopropylcyclohexanone, 3,5-diisopropylcyclohexanone, 2,4-di(t-butyl)-cyclohexanone, 3,5-di(t-butyl)cyclohexanone, 2-t-butyl-6-methylcyclohexanone, 3,3,5-trimethylcyclohexanone, 3,3,5,5-tetramethylcyclohexanone, 2,4,6-tri(t-butyl)cyclohexanone, 4-cyclopentylcyclohexanone, 4-cyclohexylcyclohexanone, 4-cyclohexyl-2-methylcyclohexanone, 2-cyclohexenone, 3-cyclohexenone, 6-bromo-2-cyclohexenone, 6-chloro-2-cyclohexenone, 2-methyl-2-cyclohexenone, 6-methyl-2-cyclohexenone, 4-isopropyl-2-cyclohexenone, 4-isobutyl-2-cyclohexenone, 4-t-butyl-2-cyclohexenone, isophorone, 2-methyl-3-cyclohexenone, 6-methyl-3-cyclohexenone 4-isopropyl-3-cyclohexenone, 4-isobutyl-3-cyclohexenone, 4-t-butyl-3-cyclohexenone and 3,3,5-trimethyl-3-cyclohexenone, 4-cyclohexyl-2-cyclohexenone, 4-cyclohexyl-3-cyclohexenone, 4-cyclopentyl-2-cyclohexenone, 4-cyclohexyl-6-methyl-2-cyclohexenone, cyclododecanone, cyclodecanone, norbornanone, norbornenone, adamantanone and other ketones derived from polycyclic hydrocarbons as well as aliphatic ketones such as, e.g., 2-hexanone, 3-hexanone, 2-heptanone, 3-heptanone, 4-heptanone, 2-octanone, 3-octanone, 2-nonanone, 3-nonanone, 2,4,8-trimethyl-4-nonanone, 2-decanone, 3-decanone, 2-undecanone, 6-undecanone, 2-methyl-4-undecanone, 2-dodecanone, 3-dodecanone and 4-dodecanone. Non-limiting examples of suitable phenol starting materials include phenol, o-cresol, m-cresol, p-cresol, o-chlorophenol, o-bromophenol, 2-ethylphenol, 2-octylphenol, 2-nonylphenol, 2,6-xylenol, 2-t-butyl-5-methylphenol, 2-t-butyl-4-methylphenol, 2,4-di(t-butyl)phenol, 2-t-butylphenol, 2-sec-butylphenol, 2-n-butylphenol, 2-cyclohexylphenol, 4-cyclohexylphenol, 2-cyclohexyl-5-methylphenol, α-decalone, and β-decalone.

It is well known in the art that this condensation chemistry can give a mixture of products such as o-alkylation of the phenol, oligomers derived from multiple alkylation of the phenol by the ketone, and acid-catalyzed rearrangement products. These impurities can either be removed or left in the material used as starting material for the cyanation reaction. In some regards these impurities can be beneficial, in that they lower the melting point of the final cyanated product. This can make it easier to prepare to formulate the cyanate by making it more soluble and reducing the tendency to crystallize. The presence of the oligomers tends to increase the viscosity of the cyanate and therefore its formulated products. This can be a beneficial or harmful property depending on the application.

By way of non-limiting example, the allylation of a bisphenol of formula (V) may be accomplished via a transcarbonation reaction using, for example, allyl methyl carbonate or a direct allylation reaction using, for example, an allyl halide, a methallyl halide and the like plus an alkaline agent and optional catalyst such as a phase transfer catalyst. Allyl methyl carbonate is usually prepared from the reaction of allyl alcohol and dimethyl carbonate to give a mixture of allyl methyl carbonate and diallyl carbonate. Both the crude mixture and the pure allyl methyl carbonate can be used as the allylating agent as well as allyl halides such as allyl chloride, allyl bromide, methallyl chloride, methallyl bromide, and the like.

A preferred process uses a transcarbonation reaction wherein allyl methyl carbonate is stoichiometrically reacted with a bisphenol of formula (V) and provides essentially total allylation of the hydroxy groups of the bisphenol to provide the corresponding allylether (allyloxy) groups. In the direct allylation reaction, an allyl halide may be stoichiometrically reacted with the hydroxy groups of the bisphenol. Depending on reaction conditions, variable amounts of Claisen rearrangement product may be observed in this reaction, resulting in mixtures of O- and C-allylated products.

A direct allylation reaction of the bisphenol of formula (V) with an allyl halide such as allyl chloride may, for example, be conducted in the presence of an alkaline agent such as an aqueous solution of an alkali metal hydroxide (e.g., NaOH). If desired, inert solvents such as, e.g., 1,4-dioxane and phase transfer catalysts such as, e.g., benzyltrialkylammonium halides or tetraalkylammonium halides can be employed. Reaction temperatures of from about 25° to about 150° C. are operable with temperatures of from about 50° to about 100° C. being preferred.

Reaction times of from about 15 minutes to about 8 hours are operable with reaction times of from about 2 hours to about 6 hours being preferred.

The reaction of a 1 to 1 mole ratio of allyl halide with the hydroxy groups of the bisphenol of formula (V) will provide an allylated bisphenol wherein the major amount (about 80 or more percent) of the hydroxy groups of the bisphenol (V) will have been converted to —O—CH₂—CH═CH₂ groups. A minor amount (about 20 percent or less) of the allyl groups will have undergone thermally induced Claisen rearrangement and will thus be present on the aromatic ring ortho and/or para to the hydroxy groups from which the rearrangement occurred. The reaction of less than a 1 to 1 mole ratio of allyl methyl carbonate in the transcarbonation reaction or of allyl halide in the direct allylation reaction with the hydroxy groups of the bisphenol will provide partial allylation of the bisphenol with some free hydroxy groups remaining. Although these partially allylated bisphenol compositions are less preferred, they are still useful as compositions of the present invention.

The present invention also provides ethylenically unsaturated monomers of formula (II):

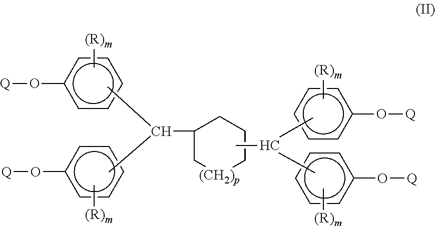

In the above formula (II), p is 0 or an integer of from 1 to about 19, e.g., up to about 14, up to about 12, or up to about 8 such as, e.g., 1, 2, 3, 4, 5, 6, and 7, with 1, 2, or 3 being preferred and 1 being particularly preferred.

The cycloaliphatic moiety shown in the above formula (II) may comprise one or more (e.g., 1, 2, 3, or 4) double bonds and/or may carry one or more (e.g., 1, 2, or 3) substituents (although the cycloaliphatic moiety will usually not comprise any double bonds and/or substituents). If more than one substituent is present, the substituents may be the same or different. Non-limiting examples of substituents which may be present on the cycloaliphatic moiety are alkyl groups, e.g., optionally substituted alkyl groups having from 1 to about 6 carbon atoms (e.g., methyl or ethyl), hydroxy, amino optionally carrying one or two alkyl groups having from 1 to about 6 carbon atoms, and halogen atoms such as, e.g., F, Cl, and Br. The alkyl groups may be substituted with, e.g., one or more halogen atoms such as, e.g., F, Cl, and Br.

The value of each m in the above formula (II) independently is 0, 1, or 2. Preferably, the values of m are identical and/or are 0 or 1.

The moieties R in the above formula (II) independently represent halogen (e.g., F, Cl, and Br, preferably Cl or Br), cyano, nitro, hydroxy, amino optionally carrying one or two alkyl groups preferably having from 1 to about 6 carbon atoms, unsubstituted or substituted alkyl preferably having from 1 to about 6 carbon atoms, unsubstituted or substituted cycloalkyl preferably having from about 5 to about 8 carbon atoms, unsubstituted or substituted alkoxy preferably having from 1 to about 6 carbon atoms, unsubstituted or substituted alkenyl preferably having from 3 to about 6 carbon atoms, unsubstituted or substituted alkenyloxy preferably having from 3 to about 6 carbon atoms, unsubstituted or substituted aryl preferably having from 6 to about 10 carbon atoms, unsubstituted or substituted aralkyl preferably having from 7 to about 12 carbon atoms, unsubstituted or substituted aryloxy preferably having from 6 to about 10 carbon atoms, and unsubstituted or substituted aralkoxy preferably having from 7 to about 12 carbon atoms.

The above alkyl groups (including the alkyl groups which may be present in the above amino group which may carry one or two alkyl groups) and alkoxy groups will often comprise from 1 to about 4 carbon atoms and in particular, 1 or 2 carbon atoms. Non-limiting specific examples of these groups include, methyl, ethyl, propyl, isopropyl, n-butyl, isobutyl, sec-butyl, tert-butyl, methoxy, ethoxy, propoxy, isopropoxy, n-butoxy, isobutoxy, sec-butoxy, and tert-butoxy. The alkyl and alkoxy groups may be substituted with one or more (e.g., 1, 2, or 3) substituents. If more than one substituent is present, the substituents may be the same or different and are preferably identical. Non-limiting examples of these substituents include halogen atoms such as, e.g., F, Cl, and Br. Non-limiting examples of substituted alkyl and alkoxy groups include CF₃, CF₃CH₂, CCl₃, CCl₃CH₂, CHCl₂, CH₂Cl, CH₂Br, CCl₃O, CHCl₂O, CH₂ClO, and CH₂BrO.

The above alkenyl and alkenyloxy groups will often comprise 3 or 4 carbon atoms and in particular, 3 carbon atoms. Non-limiting specific examples of these groups are allyl, methallyl, and 1-propenyl. The alkenyl and alkenyloxy groups may be substituted with one or more (e.g., 1, 2, or 3) substituents. If more than one substituent is present, the substituents may be the same or different and are preferably identical. Non-limiting examples of these substituents include halogen atoms such as, e.g., F, Cl, and Br.

The above aryl and aryloxy groups will often be phenyl and phenoxy groups. The aryl and aryloxy groups may be substituted with one or more (e.g., 1, 2, 3, 4, or 5) substituents. If more than one substituent is present, the substituents may be the same or different. Non-limiting examples of these substituents include hydroxy, nitro, cyano, halogen such as, e.g., F, Cl, and Br, optionally halogen-substituted alkyl having from 1 to about 6 carbon atoms, e.g., from 1 to about 4 carbon atoms (for example, methyl or ethyl), optionally halogen-substituted alkoxy having from 1 to about 6 carbon atoms, e.g., from 1 to about 4 carbon atoms (for example, methoxy or ethoxy), and amino which may optionally carry one or more alkyl groups having from 1 to about 6 carbon atoms, e.g., from 1 to about 4 carbon atoms (for example, methyl or ethyl). Non-limiting specific examples of substituted aryl and aryloxy groups include, tolyl, xylyl, ethylphenyl, chlorophenyl, bromophenyl, tolyloxy, xylyloxy, ethylphenoxy, chlorophenoxy, and bromophenoxy.

The above aralkyl and aralkoxy groups will often be benzyl, phenethyl, benzyloxy, or phenethoxy groups. These groups may be substituted (preferably on the aryl ring, if at all) with one or more (e.g., 1, 2, 3, 4, or 5) substituents. If more than one substituent is present, the substituents may be the same or different. Non-limiting examples of these substituents include hydroxy, nitro, cyano, halogen such as, e.g., F, Cl, and Br, optionally halogen-substituted alkyl having from 1 to about 6 carbon atoms, e.g., from 1 to about 4 carbon atoms (for example, methyl or ethyl), optionally halogen-substituted alkoxy having from 1 to about 6 carbon atoms, e.g., from 1 to about 4 carbon atoms (for example, methoxy or ethoxy), and amino which may optionally carry one or more alkyl groups having from 1 to about 6 carbon atoms, e.g., from 1 to about 4 carbon atoms (for example, methyl or ethyl).

The moieties Q in the above formula (II) independently represent hydrogen, HR¹C═CR¹—CH₂—, or H₂R¹C—CR¹═HC—. The moieties R¹ independently represent hydrogen or unsubstituted or substituted (preferably unsubstituted) alkyl having from 1 to about 3 carbon atoms. A preferred moiety Q is allyl. Also, it is preferred for the moieties Q to be identical and/or to be different from hydrogen. Preferably at least one of the moieties Q is different from hydrogen. Even more preferred, at least two or at least three moieties Q are different from hydrogen.

Non-limiting specific examples of the above alkyl moieties R¹ include methyl, ethyl, propyl and isopropyl. Methyl is preferred. If one or more substituents are present in these alkyl groups they may, for example, be halogen such as, e.g., F, Cl, and Br.

Non-limiting examples of the above monomers of formula (II) include dimethylcyclohexane tetraphenol tetra(allyl ether), dimethylcyclohexane tetraphenol tetra(methallyl ether), dimethylcyclohexane tetraphenol tetra(1-propenyl ether), dimethylcyclooctane tetraphenol tetra(allyl ether), dimethylcyclooctane tetraphenol tetra(methallyl ether), dimethylcyclooctane tetraphenol tetra(1-propenyl ether), partial or complete Claisen rearrangement products of dimethylcyclohexane tetraphenol tetra(allyl ether), and monomers which carry at least one substituent on at least one aromatic ring to block a Claisen rearrangement.

The monomers of the above formula (II) may be prepared, for example, by a process which comprises the condensation of a dialdehyde of a corresponding cycloalkane which comprises from about 5 to about 24 ring carbon atoms with a corresponding hydroxyaromatic (e.g., phenolic) compound (such as, e.g., phenol) at a ratio of aromatic hydroxy groups to aldehyde groups which affords a mixture of polyphenolic compounds with a polydispersity (Mw/Mn) of not higher than about 2, e.g., not higher than about 1.5, or not higher than about 1.3, and optionally subjecting the resultant mixture of polyphenolic compounds to an etherification reaction to partially or completely convert the phenolic groups which are present in the mixture into ether groups of formula HR¹C═CR¹—CH₂—O— and/or H₂R¹C—CR¹═HC—O—, wherein the moieties R¹ independently represent hydrogen or unsubstituted or substituted alkyl having from 1 to about 3 carbon atoms. This process affords the monomers of formula (II) in admixture with other monomers of similar structure but with higher and lower molecular weights (higher or lower degree of condensation).

The cycloaliphatic dialdehydes which are starting materials for the above process may be prepared by methods which are well known to those of skill in the art. By way of non-limiting example, cyclohexane (1,3 and/or 1,4)-dicarboxaldehyde can be produced, e.g., by hydroformylation of a cyclohexene carboxaldehyde, which in turn can be prepared by a Diels-Alder reaction of a conjugated diene such as, e.g., butadiene, piperylene, isoprene and chloroprene with an optionally substituted alpha,beta-unsaturated aldehyde such as, e.g., acrolein, methacrolein, crotonaldehyde or cinnamaldehyde as the dienophile. In this regard, U.S. Pat. No. 6,252,121 and Japanese patent application JP 2002-212109, the entire disclosures whereof are incorporated by reference herein, may, for example, be referred to. These (in no way limiting) reactions may be schematically represented as follows:

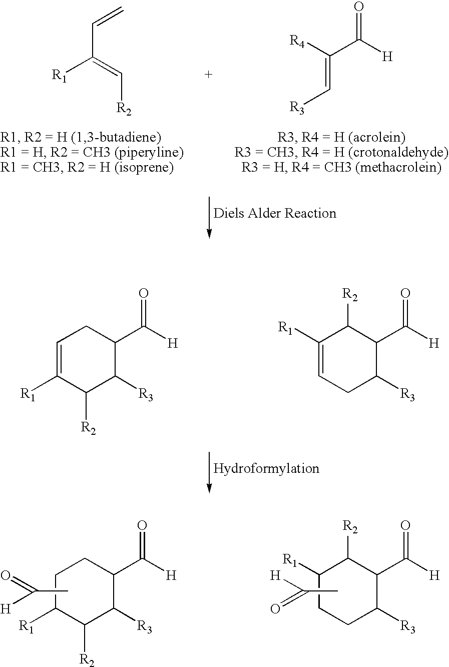

By using cyclic dienes such as, e.g., cyclopentadiene, cyclohexadiene or furan as conjugated diene in the Diels-Alder reaction, bicyclic unsaturated aldehydes may be obtained, as illustrated in the following scheme:

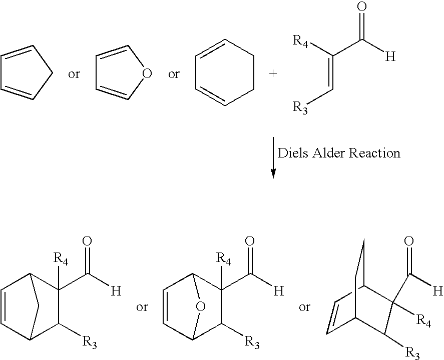

Cycloaliphatic dicarboxaldehydes may also be prepared by hydroformylation of cyclic diolefins such as, e.g., cyclooctadiene, as described in, for example U.S. Pat. No. 5,138,101 and DE 198 14 913, or by ozonolysis of bicyclic olefins such as norbornene to produce cyclopentane dicarboxaldehyde (see, e.g., Perry, J. Org. Chem., 42, 829-833, 1959). The entire disclosures of these three documents are incorporated by reference herein.

The condensation of a cycloalkane dicarboxaldehyde (or a mixture of cycloalkane dicarboxaldehydes) with, e.g., (unsubstituted) phenol affords a mixture of polyphenolic compounds which comprises a cycloalkane dicarboxaldehyde tetraphenol along with compounds with a higher (and lower) degree of condensation. The process renders it possible to produce very low polydispersity products with a high average functionality. For example, when using cyclohexane dicarboxaldehyde and phenol as starting materials, products having a weight average molecular weight (Mw) of about 930 and a number average molecular weight (Mn) of about 730 and/or an average of about 6 hydroxy groups per molecule can routinely be produced. The process uses preferably a relatively high ratio of the number of aromatic hydroxyl groups to the number of aldehyde groups (e.g., about 6:1) to keep oligomerization low. The excess hydroxyaromatic compound may then be removed, for example, by distillation.

By way of non-limiting example, the allylation of a cycloalkane tetraphenol such as, e.g., cyclohexane dicarboxaldehyde tetraphenol (and related phenolic compounds which may be present in admixture therewith) may be accomplished via a transcarbonation reaction using, for example, allyl methyl carbonate or a direct allylation reaction using, for example, an allyl halide, a methallyl halide, and the like plus an alkaline agent and an optional catalyst such as a phase transfer catalyst. Allyl methyl carbonate is usually prepared from the reaction of allyl alcohol and dimethyl carbonate to afford a mixture of allyl methyl carbonate and diallyl carbonate. Both the crude mixture and the pure allyl methyl carbonate can be used as the allylating agent as well as allyl halides such as allyl chloride, allyl bromide, methallyl chloride, methallyl bromide, and the like.

A preferred process uses a transcarbonation reaction wherein allyl methyl carbonate is stoichiometrically reacted with a cycloalkane tetraphenol and provides an essentially total allylation of the hydroxy groups of the cycloalkane tetraphenol to provide the corresponding allylether (allyloxy) groups. In the direct allylation reaction an allyl halide may be stoichiometrically reacted with the hydroxy groups of the cycloalkane tetraphenol. Depending on reaction conditions, variable amounts of Claisen rearrangement product may be observed in this reaction, resulting in mixtures of O- and C-allylated products.

A direct allylation reaction of the cycloalkane tetraphenol with an allyl halide such as allyl chloride may, for example, be conducted in the presence of an alkaline agent such as an aqueous solution of an alkali metal hydroxide (e.g., NaOH). If desired, inert solvents such as, e.g., 1,4-dioxane and phase transfer catalysts such as, e.g., benzyltrialkylammonium halides or tetraalkylammonium halides can be employed. Reaction temperatures of from about 25° to about 150° C. are operable with temperatures of from about 50° to about 100° C. being preferred.

Reaction times of from about 15 minutes to about 8 hours are operable with reaction times of from about 2 hours to about 6 hours being preferred. The reaction of a 1 to 1 mole ratio of allyl halide with the hydroxy groups of the cycloalkane tetraphenol will provide an allylated cycloalkane tetraphenol wherein the major amount (about 80 or more percent) of the hydroxyl groups of the tetraphenol will have been converted to —O—CH₂—CH═CH₂ groups. A minor amount (about 20 percent or less) of the allyl groups will have undergone a thermally induced Claisen rearrangement and will thus be present on the aromatic ring ortho and/or para to the hydroxy groups from which the rearrangement occurred. The reaction of less than a 1 to 1 mole ratio of allyl methyl carbonate in the transcarbonation reaction or of allyl halide in the direct allylation reaction with the hydroxy groups of the tetraphenol will provide a partial allylation of the tetraphenol precursor with some free hydroxy groups remaining. Although these partially allylated cycloalkane tetraphenol compositions are less preferred, they are still useful in compositions of the present invention.

The present invention also provides polymers (i.e., homo- and copolymers) and prepolymers (B-staged forms) of the ethylenically unsaturated monomers of formulae (I)/(Ia) and (II) set forth above (including the various aspects thereof).

The homopolymers or copolymers of the monomers of the above formulae (I)/(Ia) and (II) may be prepared by heating with or without a free-radical forming catalyst and/or accelerator in the presence or absence of a solvent (preferably in the absence of a solvent). Temperatures of from about 120° C. to about 350° C. are typically employed in the homopolymerization with temperatures of from about 150° C. to about 250° C. being preferred.

Suitable free radical forming catalysts which may optionally be used for the polymerization include those which are commonly employed in the free radical polymerization of ethylenically unsaturated monomers. Specific and non-limiting examples thereof include organic peroxides and hydroperoxides as well as azo and diazo compounds. Preferred examples of free radical forming catalysts include butyl peroxybenzoate, dicumyl peroxide, di-t-butylperoxide, mixtures thereof, and the like. The free radical forming catalysts may be employed, for example, at concentrations of from about 0.001 to about 2 percent by weight, based on the total weight of the monomers and/or prepolymers present.

Suitable accelerators which may optionally be used for the polymerization include those which are commonly employed in the free radical polymerization of ethylenically unsaturated monomers. Specific and non-limiting examples thereof include the metal salts of organic acids. Preferred examples of accelerators include cobalt naphthenate and cobalt octoate. The accelerators may be employed, for example, at concentrations of from about 0.001 to about 0.5 percent by weight, based on the total weight of the monomers and/or prepolymers present.

Partial homopolymerization (oligomerization or prepolymerization or B-staging) of the monomers of the above formulae (I)/(Ia) and (II) of the present invention may be effected, for example, by using lower polymerization temperatures and/or shorter polymerization reaction times than those indicated above. The curing of the prepolymerized monomers may then be completed at a later time or immediately following prepolymerization to comprise a single curing step. The progress of the (homo)polymerization can conveniently be followed by viscometry and/or infra-red spectrophotometric analysis and/or gel permeation chromatographic analysis.

The ethylenically unsaturated monomers of the present invention may be copolymerized with a variety of other monomers and/or prepolymers. In corresponding copolymerizable mixtures one or more monomers of formula (I)/(Ia) and/or (II) and/or prepolymers thereof may, for example, be present in amounts of from about 5% to about 95% by weight, e.g., from about 10% to about 90% by weight, or from about 25% to about 75% by weight, based on the total weight of the polymerizable components.

Non-limiting examples of monomers and/or prepolymers which may be copolymerized with the monomers of formula (I)/(Ia) and/or prepolymers thereof and/or with the monomers of formula (II) and/or prepolymers thereof include allyl monomers and/or prepolymers thereof. Specific and non-limiting examples of the allyl monomers and prepolymers thereof include allyl-s-triazines, allyl ethers, allyl esters, diethylene glycol bis(allylcarbonate)s, allyl phenols, and phosphorus containing allyl monomers and prepolymers thereof. These and other allyl monomers and/or prepolymers which may be copolymerized with the monomers of the present invention are described, for example, in the Encyclopedia of Polymer Science and Technology, Volume 1, pages 750 to 807 (1964) pub-lished by John Wiley and Sons, Inc., the entire disclosure of which is expressly incorporated by reference herein. Preferred allyl monomers and/or prepolymers thereof for use in the present invention include triallyl isocyanurate, 2,4,6-tris(allyloxy)-s-triazine, hexaallylmelamine, hexa(allyloxymethyl)melamine, trimethylolpropane diallyl ether, 1,2,3-methallyloxypropane, o-diallyl bisphenol A, hexamethallyldipentaerythritol, diallyl phthalate, diallyl isophthalate, diethylene glycol bis(allylcarbonate), and allyl diphenyl phosphate. The allyl monomers and/or prepolymers may be used either individually or in any combination thereof.

Further non-limiting examples of monomers and/or prepolymers which may be copolymerized with the monomers of the present invention include aromatic di- and polycyanates, aromatic di- and polycyanamides, di- and polymaleimides, bisvinylbenzyl ethers of bisphenol A or tetrabromobisphenol A, dipropargyl ethers of bisphenol A or tetrabromobisphenol A, and di- and polyglycidyl ethers (epoxy resins) such as, e.g., diglycidyl ethers of bisphenol A or bisphenol F, polyglycidyl ethers of phenol novolac or cresol novolac resins and the epoxy resins described in the co-assigned application entitled “POLYPHENOLIC COMPOUNDS AND EPOXY RESINS COMPRISING CYLCOALIPHATIC MOIETIES AND PROCESS FOR THE PRODUCTION THEREOF”, filed concurrently herewith (Attorney Docket No. 65221), the entire disclosure whereof is expressly incorporated by reference herein.

Of course, it is possible to copolymerize the monomers of the present invention and/or prepolymers thereof also with other components such as, e.g., one or more of (a) at least one compound which contains in the same molecule both a cyanate or cyanamide group and a polymerizable ethylenically unsaturated group; (b) at least one compound which contains in the same molecule both a 1,2-epoxide group and a polymerizable ethylenically unsaturated group; (c) at least one compound which contains in the same molecule both a maleimide group and a cyanate group; (d) at least one polyamine, (e) at least one polyphenol, etc.

Non-limiting examples of dicyanates which may be copolymerized with the monomers of the present invention and/or prepolymers thereof include dicyanate compounds of the following formula (III) and/or prepolymers thereof:

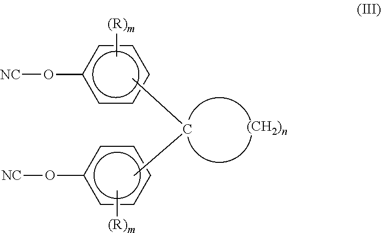

In the above formula (III), n, m and R and the cycloaliphatic moiety may have the same meanings (including exemplary and preferred meanings) as those set forth above with respect to formula (Ia). The compounds of formula (III) are more fully described in the co-assigned application entitled “AROMATIC DICYANATE COMPOUNDS WITH HIGH ALIPHATIC CARBON CONTENT”, filed concurrently herewith (Attorney Docket No. 66499), the entire disclosure of which is expressly incorporated by reference herein.

Further non-limiting examples of monomers (prepolymers) which may be copolymerized with the monomers (prepolymers) of the present invention include cyanate compounds of the above formula (III) wherein one of the cyano groups is replaced by an ethylenically unsaturated group such as, e.g., a group of formula HR¹C═CR¹—CH₂—O— or H₂R¹C—CR¹═HC—O— wherein the moieties R¹ are defined as set forth above with respect to formula (I)/(Ia).

Further non-limiting examples of cyanates which may be polymerized with the monomers of the present invention and/or prepolymers thereof include compounds of the following formula (IV) and/or prepolymers thereof:

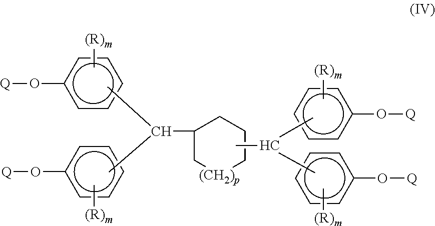

In the above formula (IV), p, m, and R and the cycloaliphatic moiety may have the same meanings (including exemplary and preferred meanings) as those set forth above with respect to formula (II). Further, at least two of the moieties Q represent —CN and the remaining moieties Q preferably represent hydrogen. For example, at least three or all four moieties Q may represent —CN. Compounds of formula (IV) are more fully described in co-assigned application entitled “AROMATIC POLYCYANATE COMPOUNDS AND PROCESS FOR THE PRODUCTION THEREOF”, filed concurrently herewith (Attorney Docket No. 66500), the entire disclosure of which is expressly incorporated by reference herein.

Further non-limiting examples of compounds which may be copolymerized with the monomers and/or prepolymers of the present invention include compounds of the above formula (IV) and prepolymers thereof wherein at least one of the moieties Q represents —CN and at least one other moiety Q represents a group of formula HR¹C═CR¹—CH₂— or H₂R¹C—CR¹═HC—, in which the moieties R¹ are as set forth above with respect to formulae (I) and (II). For example, two of the moieties Q in formula (IV) may represent —CN and one or two of the remaining moieties Q may represent a group of formula HR¹C═CR¹—CH₂— or H₂R¹C—CR¹═HC—.

The (co)polymerizable mixtures of the present invention and the products made therefrom respectively, may further comprise one or more other substances such as, e.g., one or more additives which are commonly present in polymerizable mixtures and products made therefrom. Non-limiting examples of such additives include polymerization catalysts, co-curing agents, flame retardants, synergists for flame retardants, solvents, fillers, glass fibers, adhesion promoters, wetting aids, dispersing aids, surface modifiers, thermoplastic resins, and mold release agents.

Non-limiting examples of co-curing agents for use in the present invention include dicyandiamide, substituted guanidines, phenolics, amino compounds, benzoxazine, anhydrides, amido amines, and polyamides.

Non-limiting examples of catalysts for use in the present invention include transition metal complexes, imidazoles, phosphonium salts, phosphonium complexes, tertiary amines, hydrazides, “latent catalysts” such as Ancamine 2441 and K61B (modified aliphatic amines available from Air Products), Ajinomoto PN-23 or MY-24, and modified ureas.

Non-limiting examples of flame retardants and synergists for use in the present invention include phosphorus containing molecules (DOP—epoxy reaction product), adducts of DOPO (6H-dibenz[c,e][1,2]oxaphosphorin-6-oxide), magnesium hydrate, zinc borate, and metallocenes.

Non-limiting examples of solvents for use in the present invention (for example, for improving processability) include acetone, methylethyl ketone, and Dowanol® PMA (propylene glycol methyl ether acetate available from Dow Chemical Company).

Non-limiting examples of fillers for use in the present invention include functional and non-functional particulate fillers with a particle size range of from about 0.5 nm to about 100 μm. Specific examples thereof include silica, alumina trihydrate, aluminum oxide, metal oxides, carbon nanotubes, silver flake or powder, carbon black, and graphite.

Non-limiting examples of adhesion promoters for use in the present invention include modified organosilanes (epoxidized, methacryl, amino, allyl, etc.), acetylacetonates, sulfur containing molecules, titanates, and zirconates.

Non-limiting examples of wetting and dispersing aids for use in the present invention include modified organosilanes such as, e.g., Byk 900 series and W 9010, and modified fluorocarbons.

Non-limiting examples of surface modifiers for use in the present invention include slip and gloss additives, a number of which are available from Byk-Chemie, Germany.

Non-limiting examples of thermoplastic resins for use in the present invention include reactive and non-reactive thermoplastic resins such as, e.g., polyphenylsulfones, polysulfones, polyethersulfones, polyvinylidene fluoride, polyetherimides, polyphthalimides, polybenzimidazoles, acrylics, phenoxy resins, and polyurethanes.

Non-limiting examples of mold release agents for use in the present invention include waxes such as, e.g., carnauba wax.

The monomers of the present invention are useful, inter alia, as thermosettable comonomers for the production of printed circuit boards and materials for integrated circuit packaging (such as IC substrates). They are especially useful for formulating matrix resins for high speed printed circuit boards, integrated circuit packaging, and underfill adhesives. As a comonomer, they may also be used to adjust the amount of hydrocarbon in a thermoset matrix.

Additionally, the monomers of the present invention may be homopolymerized, for example using a free-radical forming catalyst and/or accelerator, to produce rigid, glassy polymers with an anticipated high degree of toughness, corrosion resistance, and moisture resistance. The utility for these homopolymers may be in the same applications that are served by poly[diethylene glycol bis(allyl carbonate)], also known as CR-39, and includes optical lenses, but with enhanced mechanical properties.

### Example 1

### Synthesis of Bis(Allyl Ether) of 1,1-Bis(4-hydroxyphenyl)cyclododecane

Allyl alcohol (101.58 grams, 1.75 moles), dimethyl carbonate (157.55 grams, 1.75 moles) and sodium methoxide catalyst (0.18 gram, 0.065 percent by weight) were added to a 500 milliliter, 3 neck, round bottom glass reactor and maintained at room temperature (23° C.) with stirring under a nitrogen atmosphere. The reactor was additionally outfitted with a chilled condenser, a thermometer, magnetic stiffing, and a thermostatically controlled heating mantle. An equilibrium mixture of allylmethyl carbonate, diallyl carbonate and methanol was rapidly formed concurrent with cooling of the reactor contents to 15.5° C. After 13 minutes 1,1-bis(4-hydroxyphenyl)cyclododecane (28.31 grams, 0.1606 equivalent of hydroxy groups) was added to the reactor, followed by a mixture of triphenylphosphine (0.56 gram, 0.204 percent by weight) and 5% palladium on carbon (0.38 gram, 0.127 percent by weight). The 1,1-bis(4-hydroxyphenyl)cyclododecane assayed 99.76 area % via high pressure liquid chromatographic (HPLC) analysis with the balance consisting of 2 minor components (0.09 and 0.15 area %). Heating commenced and over the next 127 minutes the reaction temperature reached 79-80° C. The reaction mixture was maintained for 8 hours at 77.5-80° C. and then cooled to room temperature and vacuum filtered through a bed of diatomaceous earth packed on a medium fritted glass funnel. The recovered filtrate was rotary evaporated at a maximum oil bath temperature of 100° C. and to a vacuum of 1.7 mm Hg pressure to provide a transparent, light yellow colored, liquid (35.04 grams) which became a tacky solid at room temperature.

HPLC analysis revealed the presence of 96.78 area % allyl ether of 1,1-bis(4-hydroxyphenyl)cyclododecane with the balance as a single minor component (3.22 area %). The single minor component was removed by dissolving the product in dichloromethane (100 milliliters) and passing the resultant solution through a 2 inch deep by 1.75 inch diameter bed of silica gel (230-400 mesh particle size, 60 angstrom mean pore size, 550 m²/gram surface dimension) supported on a medium fritted glass funnel. After elution from the silica gel bed with additional dichloromethane, a yellow band remained in the region of the origin. Rotary evaporation provided 33.98 grams (98.94% isolated yield) of pale yellow colored tacky solid.

HPLC analysis revealed the presence of 99.57 area % allyl ether of 1,1-bis(4-hydroxyphenyl)cyclododecane with the balance as 2 minor components (0.22 and 0.21 area %). Infrared spectrophotometric analysis of a film sample of the product on a KBr plate revealed peaks in the range expected for unsaturated C—H stretch (3032, 3058, 3081 cm⁻¹), saturated C—H stretch (2862, 2934 cm⁻¹ [shoulder present on both]), C═C stretch (1581, 1607 cm⁻¹), C—O stretch (1026 cm⁻¹), and CH═CH₂ deformation (924, 998 cm⁻¹), accompanied by total absence of hydroxyl group absorbance thus confirming full conversion of the phenolic hydroxyl groups to allyl ether groups.

### Example 2

### Thermally Induced Homopolymerization of Bis(Allyl Ether) of 1,1-Bis(4-hydroxyphenyl)cyclododecane

Differential scanning calorimetry (DSC) analysis of a portion (10.00 milligrams) of the bis(allyl ether) of 1,1-bis(4-hydroxyphenyl)cyclododecane from Example 1 was conducted using a rate of heating of 5° C. per minute from 25° C. to 400° C. under a stream of nitrogen flowing at 35 cubic centimeters per minute. A pair of exotherms attributed to homopolymerization of the allyl groups were observed with a 181.5° C. onset, 253.4° C. maximum, and a 283.9° C. endpoint accompanied by an enthalpy of 243.4 joules per gram for the initial exotherm and a 284.8° C. onset, 351.3° C. maximum, and a 396.2° C. endpoint accompanied by an enthalpy of 181.5 joules per gram for the second exotherm. The homopolymer recovered from the DSC analysis was a transparent, amber colored, rigid solid.

### Comparative Experiment A

### Synthesis of Bis(Allyl Ether) of Isopropylidene Diphenol

Allyl alcohol (101.58 grams, 1.75 moles), dimethyl carbonate (157.55 grams, 1.75 moles) and sodium methoxide catalyst (0.18 gram, 0.065 percent by weight) were added to a 500 milliliter, 3 neck, round bottom glass reactor and maintained at room temperature (23° C.) with stiffing under a nitrogen atmosphere. The reactor was additionally outfitted with a chilled condenser, a thermometer, magnetic stirring, and a thermostatically controlled heating mantle. An equilibrium mixture of allylmethyl carbonate, diallyl carbonate and methanol was rapidly formed concurrent with cooling of the reactor contents to 15.5° C. After 13 minutes, isopropylidene diphenol (=bisphenol A, 18.33 grams, 0.1606 equivalent of hydroxy groups), was added to the reactor followed by a mixture of triphenylphosphine (0.56 gram, 0.204 percent by weight) and 5% palladium on carbon (0.38 gram, 0.127 percent by weight). The isopropylidene diphenol assayed 99.72 area % via HPLC analysis with the balance consisting of 2 minor components (0.09 and 0.19 area %). Heating commenced and over the next 101 minutes, the reaction temperature reached 78° C. The reaction mixture was maintained for 8 hours at 78° C. and then cooled to room temperature and vacuum filtered through a bed of diatomaceous earth packed on a medium fitted glass funnel. The recovered filtrate was rotary evaporated at a maximum oil bath temperature of 100° C. and to a vacuum of 2.9 mm Hg pressure to provide a transparent, amber colored, liquid (25.21 grams) which remained liquid at room temperature.

HPLC analysis revealed the presence of 95.25 area % allyl ether of isopropylidene diphenol with the balance as 12 minor components (ranging from 0.05 to 2.13 area %). The single minor component comprising 2.13 area % along with other minor components was removed by dissolving the product in dichloromethane (75 milliliters) and passing the resultant solution through a 2 inch deep by 1.75 inch diameter bed of silica gel (230-400 mesh particle size, 60 angstrom mean pore size, 550 m²/gram surface dimension) supported on a medium fritted glass funnel. After elution from the silica gel bed with additional dichloromethane, a yellow band remained in the region of the origin. Rotary evaporation provided 23.32 grams (94.17% isolated yield) of light yellow colored liquid.

HPLC analysis revealed the presence of 99.51 area % allyl ether of isopropylidene diphenol with the balance as 3 minor components (0.13, 0.05, and 0.31 area %). Infrared spectrophotometric analysis of a film sample of the product on a KBr plate revealed peaks in the range expected for unsaturated C—H stretch (3039, 3061, 3083 cm⁻¹), saturated C—H stretch (2870, 2931[shoulder present], 2966 cm⁻¹), C═C stretch (1581, 1608 cm⁻¹), C—O stretch (1025 cm⁻¹), and CH═CH₂ deformation (926, 998 cm⁻¹), accompanied by total absence of hydroxyl group absorbance thus confirming full conversion of the phenolic hydroxyl groups to allyl ether groups.

### Comparative Experiment B

### Thermally Induced Homopolymerization of Bis(Allyl Ether) of Isopropylidene Diphenol

DSC analysis of a portion (11.20 milligrams) of the bis(allyl ether) of isopropylidene diphenol from Comparative Experiment A was conducted using a rate of heating of 5° C. per minute from 25° C. to 400° C. under a stream of nitrogen flowing at 35 cubic centimeters per minute. A pair of exotherms attributed to homopolymerization of the allyl groups were observed with a 201.4° C. onset, 253.4° C. maximum, and a 278.6° C. endpoint accompanied by an enthalpy of 267.1 joules per gram for the initial exotherm and a 278.6° C. onset, 351.2° C. maximum, and a 387.2° C. endpoint accompanied by an enthalpy of 212.2 joules per gram for the second exotherm. The homopolymer recovered from the DSC analysis was a transparent, amber colored, rigid solid.

| | | | Exotherm | Exotherm | |
|---|---|---|---|---|---|
| | | Exotherm | Maximum | Endpoint | Enthalpy |
| Designation | Monomer Used | Onset (° C.) | (° C.) | (° C.) | (joules/g) |
| Example 2 | 100% bis(allyl | 181.5 | 253.4 | 283.9 | 243.4 |
| | ether) of 1,1-bis(4- | 284.8 | 351.3 | 396.2 | 181.5 |
| | hydroxyphenyl)cyclododecane | | | | |
| Comparative | 100% bis(allyl | 201.4 | 253.4 | 278.6 | 267.1 |
| Experiment B | ether) of 4,4′- | 278.6 | 351.2 | 387.2 | 212.2 |
| | isopropylidene | | | | |
| | diphenol | | | | |

### Reference Example 1

### Synthesis of 1,1-Bis(4-cyanatophenyl)cyclododecane

A 250 milliliter, three neck, glass, round bottom reactor was charged with 1,1-bis(4-hydroxyphenyl)cyclododecane (17.63 grams, 0.10 hydroxyl equivalent) and acetone (125 milliliters, 7.09 milliliter per gram of bisphenol). The reactor was additionally equipped with a condenser (maintained at 0° C.), a thermometer, an overhead nitrogen inlet (1 LPM N₂ used), and magnetic stirring. Stirring commenced to give a solution at 21.5° C. Cyanogen bromide (11.12 grams, 0.105 mole, 1.05:1 cyanogen bromide:hydroxyl equivalent ratio) was added to the solution and immediately dissolved therein. A dry ice-acetone bath for cooling was placed under the reactor followed cooling and equilibration of the stirred solution at −5° C. Triethylamine (10.17 grams, 0.1005 mole, 1.005 triethylamine: hydroxyl equivalent ratio) was added using a syringe in aliquots that maintained the reaction temperature at −5 to 0° C. The total addition time for the triethylamine was 30 minutes. Addition of the initial aliquot of triethylamine induced haziness in the stirred solution with further additions inducing formation of a white slurry of triethylamine hydrobromide.

After 8 minutes of post-reaction at −5 to 0.5° C. high pressure liquid chromatographic (HPLC) analysis of a sample of the reaction product revealed the presence of 0.68 area percent unreacted 1,1-bis(4-hydroxyphenyl)cyclododecane, 4.43 area % monocyanate and 93.98 area % dicyanate with the balance as 7 minor peaks. After a cumulative 45 minutes of postreaction at −5 to 0° C. HPLC analysis of a sample of the reaction product revealed the presence of 0.84 area percent unreacted 1,1-bis(4-hydroxyphenyl)cyclododecane, 5.34 area % monocyanate and 93.51 area % dicyanate with the balance as one minor peak.

After a cumulative 101 minutes of post-reaction, the product slurry was added to a beaker of magnetically stirred deionized water (1.5 liters) providing an aqueous slurry. After 5 minutes of stirring, gravity filtration of the aqueous slurry through filter paper recovered the white powder product. The product from the filter paper was rinsed into a beaker using deionized water to a total volume of 200 milliliters, followed by the addition of dichloromethane (200 milliliters). A solution formed in the dichloromethane layer. The mixture was added to a separatory funnel, thoroughly mixed, allowed to settle, and then the dichloromethane layer recovered, with the aqueous layer discarded to waste. The dichloromethane solution was added back into the separatory funnel and extracted with fresh deionized water (200 milliliters) two additional times.

The resultant hazy dichloromethane solution was dried over granular anhydrous sodium sulfate (5 grams) to give a clear solution which was then passed through a bed of anhydrous sodium sulfate (25 grams) supported on a 60 milliliter, medium fritted glass funnel attached to a side arm vacuum flask. The clear filtrate was rotary evaporated using a maximum oil bath temperature of 50° C. until the vacuum was <3.5 mm Hg. A total of 19.81 grams (98.43% uncorrected, isolated yield) of white, crystalline product was recovered. HPLC analysis of a sample of the product revealed the presence of 0.47 area percent unreacted 1,1-bis(4-hydroxyphenyl)cyclododecane, 3.09 area % monocyanate and 96.44 area % dicyanate.

### Reference Example 2

### Synthesis and Recrystallization to Produce High Purity 1,1-Bis(4-cyanatophenyl)cyclododecane

The synthesis of dicyanate of 1,1-bis(4-hydroxyphenyl)cyclododecane of Reference Example 1 was repeated, but with a 2-fold increase in scale. The 38.86 grams of recovered product assayed 0.69 area percent unreacted 1,1-bis(4-hydroxyphenyl)cyclododecane, 3.91 area % monocyanate and 95.40 area % dicyanate by HPLC analysis. Recrystallization was performed by forming a solution in boiling acetone (50 milliliters), then holding for 24 hours at 23° C. The acetone solution was removed from the crystalline product via decantation. HPLC analysis of a portion of the damp crystalline product revealed the presence of no detectable unreacted 1,1-bis(4-hydroxyphenyl)cyclododecane, 1.02 area % monocyanate and 98.98 area % dicyanate. A second recrystallization of the damp crystalline product from acetone (40 milliliters) followed by drying in the vacuum oven at 50° C. for 48 hours provided 20.12 grams of brilliant white product with no detectable unreacted 1,1-bis(4-hydroxyphenyl)cyclododecane, 0.42 area % monocyanate and 99.58 area % dicyanate by HPLC analysis. Combination of the acetone solution decants from the two recrystallizations followed by concentration of the solution to a volume of 28 milliliters yielded a second crop of brilliant white product (8.39 grams) with a trace (non-integratable) of unreacted 1,1-bis(4-hydroxyphenyl)cyclododecane, 2.28 area % monocyanate and 97.72 area % dicyanate by HPLC analysis.

### Example 3

Thermally Induced Copolymerization of Bis(Allyl Ether) of 1,1-Bis(4-hydroxyphenyl)cyclododecane (25% wt.) and 1,1-Bis(4-cyanatophenyl)cyclododecane (75% wt.)

1,1-Bis(4-cyanatophenyl)cyclododecane (0.5034 gram, 75% wt.) and bis(allyl ether) of 1,1-bis(4-hydroxyphenyl)cyclododecane (0.1678 gram, 25% wt.) from Example 1 were weighed into a glass vial to which dichloromethane (1.5 milliliters) was added. HPLC analysis of the 1,1-bis(4-cyanatophenyl)cyclododecane revealed 99.44 area % dicyanate and 0.56 area % monocyanate. Shaking the vial provided a solution which was added to an aluminum tray. Devolatilization conducted in a vacuum oven at 40° C. for 30 minutes removed the dichloromethane giving a homogeneous blend. DSC analysis of portions (9.70 and 10.00 milligrams) of the blend was conducted using a rate of heating of 5° C. per minute from 25° C. to 400° C. under a stream of nitrogen flowing at 35 cubic centimeters per minute.

An endotherm was observed with an average 99.0° C. onset (98.07 and 99.96° C.), 118.8° C. minimum (118.72° C. and 118.93° C.), and 126.5° C. endpoint (124.61° C. and 128.40° C.) accompanied by an enthalpy of 11.5 joules per gram (10.13 and 12.76 joules per gram) (individual values in parenthesis). An exotherm attributed to copolymerization of the allyl and cyanate groups (plus any homopolymerization) was observed with an average 172.2° C. onset (170.58° C. and 173.90° C.), 249.1° C. maximum (248.30° C. and 249.80° C.), and 292.9° C. endpoint (289.54° C. and 296.18° C.) accompanied by an enthalpy of 487.1 joules per gram (474.9 and 499.2 joules per gram) (individual values in parenthesis). The copolymer recovered from the DSC analysis was a transparent, amber colored, rigid solid.

### Comparative Experiment C

### Thermally Induced Copolymerization of Bis(Allyl Ether) of Isopropylidene Diphenol (25% wt.) and 1,1-Bis(4-cyanatophenyl)cyclododecane (75% wt.)

1,1-Bis(4-cyanatophenyl)cyclododecane (0.4004 gram, 75% wt.) and bis(allyl ether) of isopropylidene diphenol (0.1335 gram, 25% wt.) from Comparative Experiment A were weighed into a glass vial to which dichloromethane (1.5 milliliters) was added. HPLC analysis of the 1,1-bis(4-cyanatophenyl)cyclododecane revealed 99.44 area % dicyanate and 0.56 area % monocyanate. Shaking the vial provided a solution which was added to an aluminum tray. Devolatilization conducted in a vacuum oven at 40° C. for 30 minutes removed the dichloromethane giving a homogeneous blend.

DSC analysis of portions (10.00 and 10.20 milligrams) of the blend was conducted using a rate of heating of 5° C. per minute from 25° C. to 400° C. under a stream of nitrogen flowing at 35 cubic centimeters per minute. An endotherm was observed with an average 69.6° C. onset (67.73° C. and 71.52° C.), 114.4° C. minimum (113.96° C. and 114.81° C.), and 127.7° C. endpoint (125.08° C. and 130.29° C.) accompanied by an enthalpy of 40.3 joules per gram (38.86 and 41.78 joules per gram) (individual values in parenthesis). An exotherm attributed to a copolymerization of the allyl and cyanate groups (plus any homopolymerization) was observed with an average 173.0° C. onset (172.95° C. and 172.95° C.), 252.5° C. maximum (250.70° C. and 254.22° C.), and 291.2° C. endpoint (289.54° C. and 292.86° C.) accompanied by an enthalpy of 512.5 joules per gram (510.4 and 514.6 joules per gram) (individual values in parenthesis). The copolymer recovered from the DSC analysis was a transparent, amber colored, rigid solid.

| | | | Exotherm | Exotherm | |
|---|---|---|---|---|---|
| | | Exotherm | Maximum | Endpoint | Enthalpy |
| Designation | Monomer Used | Onset (° C.) | (° C.) | (° C.) | (joules/g) |
| Example 3 | 25% bis(allyl | 99.0^{a} | 118.8^{a} | 126.5^{a} | 11.5^{a} |
| | ether) of 1,1- | 172.2 | 249.1 | 292.9 | 487.1 |
| | bis(4-hydroxyphenyl)cyclododecane/ | | | | |
| | 75% 1,1-bis(4- | | | | |
| | cyanatophenyl) | | | | |
| | cyclododecane | | | | |
| Comparative | 25% bis(allyl | 69.6^{a} | 114.4^{a} | 127.7^{a} | 40.3^{a} |
| Experiment B | ether) of 4,4′- | 173.0 | 252.5 | 291.1 | 512.2 |
| | isopropylidene | | | | |
| | diphenol/ | | | | |
| | 75% 1,1-bis(4- | | | | |
| | cyanatophenyl) | | | | |
| | cyclododecane | | | | |

^{a}endothermic event

### Example 4

### Glass Transition Temperature of Copolymer of Bis(Allyl Ether) of 1,1-Bis(4-hydroxyphenyl)cyclododecane (25% wt.) and 1,1-Bis(4-cyanatophenyl)cyclododecane (75% wt.)

Curing of the remaining blend from Example 3 was completed in an oven using the following curing schedule: 150° C. for 1 hour, 200° C. for 1 hour, 250° C. for 1 hour. DSC analysis of portions (28.2 and 35.0 milligrams) of the cured product gave an average glass transition temperature of 214.3° C. (212.85° C. and 215.83° C.) (individual values in parenthesis).

### Comparative Experiment D

### Glass Transition Temperature of Copolymer of Bis(Allyl Ether) of Isopropylidene Diphenol (25% wt.) and 1,1-Bis(4-cyanatophenyl)cyclododecane (75% wt.)

Curing of the remaining blend from Comparative Experiment C was completed in an oven using the following curing schedule: 150° C. for 1 hour, 200° C. for 1 hour, 250° C. for 1 hour. DSC analysis of portions (31.0 and 29.7 milligrams) of the cured product gave an average glass transition temperature of 184.48° C. (184.14° C. and 184.82° C.) (individual values in parenthesis).

| Designation | Monomer Used | Tg (° C.) |
|---|---|---|
| Example 4 | 25% bis(allyl ether) of 1,1-bis(4- | 214.3 |
| | hydroxyphenyl)cyclododecane/ | |
| | 75% 1,1-bis(4-cyanatophenyl)- | |
| | cyclododecane | |
| Comparative | 25% bis(allyl ether) of 4,4′- | 184.48 |
| Experiment D | isopropylidene diphenol/ | |
| | 75% 1,1-bis(4-cyanatophenyl)- | |
| | cyclododecane | |

### Example 5

### Thermally Induced Copolymerization of Bis(Allyl Ether) of 1,1-Bis(4-hydroxyphenyl)cyclododecane (50% wt.) and 1,1-Bis(4-cyanatophenyl)cyclododecane (50% wt.)

1,1-Bis(4-cyanatophenyl)cyclododecane (0.2978 gram, 50% wt.) and bis(allyl ether) of 1,1-bis(4-hydroxyphenyl)cyclododecane (0.2978 gram, 50% wt.) from Example 1 were weighed into a glass vial to which dichloromethane (1.5 milliliters) was added. HPLC analysis of the 1,1-bis(4-cyanatophenyl)cyclododecane revealed 99.44 area % dicyanate and 0.56 area % monocyanate. Shaking the vial provided a solution which was added to an aluminum tray. Devolatilization conducted in a vacuum oven at 40° C. for 30 minutes removed the dichloromethane giving a homogeneous blend.

DSC analysis of portions (9.70 and 10.70 milligrams) of the blend was conducted using a rate of heating of 5° C. per minute from 25° C. to 400° C. under a stream of nitrogen flowing at 35 cubic centimeters per minute. No endotherm was observed. An exotherm attributed to copolymerization of the allyl and cyanate groups (plus any homopolymerization) was observed with an average 173.7° C. onset (171.05° C. and 176.27° C.), 246.5° C. maximum (245.96° C. and 247.01° C.), and 282.0° C. endpoint (281.01° C. and 282.91° C.) accompanied by an enthalpy of 414.2 joules per gram (403.2 and 425.1 joules per gram) (individual values in parenthesis). The copolymer recovered from the DSC analysis was a transparent, amber colored, rigid solid.

### Comparative Experiment E

### Thermally Induced Copolymerization of Bis(Allyl Ether) of Isopropylidene Diphenol (50% wt.) and 1,1-Bis(4-cyanatophenyl)cyclododecane (50% wt.)

1,1-Bis(4-cyanatophenyl)cyclododecane (0.2945 gram, 50% wt.) and bis(allyl ether) of 4,4′-isopropylidene diphenol (0.2945 gram, 50% wt.) from Comparative Experiment A were weighed into a glass vial to which dichloromethane (1.5 milliliters) was added. HPLC analysis of the 1,1-bis(4-cyanatophenyl)cyclododecane revealed 99.44 area % dicyanate and 0.56 area % monocyanate. Shaking the vial provided a solution which was added to an aluminum tray. Devolatilization conducted in the vacuum oven at 40° C. for 30 minutes removed the dichloromethane giving a homogeneous blend.

DSC analysis of portions (11.20 and 11.80 milligrams) of the blend was conducted using a rate of heating of 5° C. per minute from 25° C. to 400° C. under a stream of nitrogen flowing at 35 cubic centimeters per minute. An endotherm was observed with an average 71.53° C. onset (68.21° C. and 74.84° C.), 101.13° C. minimum (99.49° C. and 102.76° C.), and 116.55° C. endpoint (115.60° C. and 117.50° C.) accompanied by an enthalpy of 15.17 joules per gram (12.03 and 18.30 joules per gram) (individual values in parenthesis). An exotherm attributed to a copolymerization of the allyl and cyanate groups (plus any homopolymerization) was observed with an average 186.93° C. onset (186.69° C. and 187.17° C.), 246.60° C. maximum (241.78° C. and 251.42° C.), and 282.20° C. endpoint (280.54° C. and 283.85° C.) accompanied by an enthalpy of 446.9 joules per gram (402.4 and 491.3 joules per gram) (individual values in parenthesis). A second exotherm attributed to homopolymerization of allyl groups was observed with an average 293.10° C. onset (292.38° C. and 293.81° C.), 352.98° C. maximum (350.00° C. and 355.95° C.), and 392.86° C. endpoint (392.86° C. and 392.86° C.) accompanied by an enthalpy of 60.9 joules per gram (51.78 and 70.10 joules per gram) (individual values in parenthesis). The copolymer recovered from the DSC analysis was a transparent, amber colored, rigid solid.

| | | | Exotherm | Exotherm | |
|---|---|---|---|---|---|
| | | Exotherm | Maximum | Endpoint | Enthalpy |
| Designation | Monomer Used | Onset (° C.) | (° C.) | (° C.) | (joules/g) |
| Example 5 | 50% bis(allyl | ^{a}173.7 | 246.5 | 282.0 | 414.2 |
| | ether) of 1,1- | | | | |
| | bis(4-hydroxy- | | | | |
| | phenyl)cyclo- | | | | |
| | dodecane/ | | | | |
| | 50% 1,1-bis(4- | | | | |
| | cyanatophenyl) | | | | |
| | cyclododecane | | | | |
| Comparative | 50% bis(allyl | 71.53^{b} | 101.13^{b} | 116.55^{b} | 15.17^{b} |
| Experiment E | ether) of 4,4′- | 186.93 | 246.60 | 282.20 | 446.9 |
| | isopropylidene | 293.10 | 352.98 | 392.86 | 60.9 |
| | diphenol/ | | | | |
| | 50% 1,1-bis(4- | | | | |
| | cyanatophenyl) | | | | |
| | cyclododecane | | | | |

^{a}no endothermic event observed
^{b}endothermic event

### Example 6

### Glass Transition Temperature of Copolymer of Bis(Allyl Ether) of 1,1-Bis(4-hydroxyphenyl)cyclododecane (50% wt.) and 1,1-Bis(4-cyanatophenyl)cyclododecane (50% wt.)

Curing of the remaining blend from Example 5 was completed in an oven using the following curing schedule: 150° C. for 1 hour, 200° C. for 1 hour, 250° C. for 1 hour. DSC analysis of portions (33.8 and 34.3 milligrams) of the cured product gave residual exothermicity at >260° C. After a second scanning an average glass transition temperature of 144.57° C. (140.98° C. and 148.15° C.) (individual values in parenthesis) was measured. A third scanning was completed since residual exothermicity was observed at >330° C. An average glass transition temperature of 160.03° C. (159.52° C. and 160.53° C.) with no residual exothermicity observed.

### Comparative Experiment F

### Glass Transition Temperature of Copolymer of Bis(Allyl Ether) of Isopropylidene Diphenol (50% wt.) and 1,1-Bis(4-cyanatophenyl)cyclododecane (50% wt.)

Curing of the remaining blend from Comparative Experiment E was completed in an oven using the following curing schedule: 150° C. for 1 hour, 200° C. for 1 hour, 250° C. for 1 hour. DSC analysis of portions (33.4 and 35.4 milligrams) of the cured product gave residual exothermicity at >260° C. After a second scanning an average glass transition temperature of 121.52° C. (118.65° C. and 124.38° C.) (individual values in parenthesis) was measured with no residual exothermicity observed. A third scanning was conducted with no change in the glass transition temperature.

| Designation | Monomer Used | Tg (° C.) |
|---|---|---|
| Example 6 | 50% bis(allyl ether) of 1,1-bis(4- | 144.57^{a} |
| | hydroxyphenyl)cyclododecane/ | 160.03^{b} |
| | 50% 1,1-bis(4-cyanatophenyl)- | |
| | cyclododecane | |
| Comparative | 50% bis(allyl ether) of 4,4′- | 121.52^{c} |
| Experiment F | isopropylidene diphenol/ | |
| | 50% 1,1-bis(4-cyanatophenyl)- | |
| | cyclododecane | |

^{a}Tg after second scanning
^{b}Tg after third scanning
^{c}Tg after second scanning, unchanged after third scanning

### Example 7

### Thermally Induced Copolymerization of Bis(Allyl Ether) of 1,1-Bis(4-hydroxyphenyl)cyclododecane (25% wt.) and Dicyanate of Isopropylidene Diphenol (75% wt.)

Dicyanate of 4,4′-isopropylidenediphenol (2.5518 gram, 75% wt.) and bis(allyl ether) of 1,1-bis(4-hydroxyphenyl)cyclododecane (0.8506 gram, 25% wt.) from Example 1 were weighed into a glass vial. HPLC analysis of the dicyanate of 4,4′-isopropylidenediphenol revealed 100 area % dicyanate. Gentle heating (did not exceed 75° C.) and mixing by swirling the contents of the vial provided a solution.

DSC analysis of portions (12.80 and 14.10 milligrams) of the blend was conducted using a rate of heating of 5° C. per minute from 25° C. to 400° C. under a stream of nitrogen flowing at 35 cubic centimeters per minute. An endotherm was observed (in only one sample) with a 30.29° C. onset, 74.59° C. minimum, and 81.00° C. endpoint accompanied by an enthalpy of 66.59 joules per gram. An exotherm attributed to a copolymerization of the allyl and cyanate groups (plus any homopolymerization) was observed with an average 196.65° C. onset (192.86° C. and 200.44° C.), 252.51° C. maximum (249.33° C. and 255.68° C.), and 289.78° C. endpoint (286.70° C. and 292.86° C.) accompanied by an enthalpy of 651.8 joules per gram (615.2 and 688.4 joules per gram) (individual values in parenthesis). The copolymer recovered from the DSC analysis was a transparent, amber colored, rigid solid.

### Comparative Experiment G

### Thermally Induced Copolymerization of Bis(Allyl Ether) of Isopropylidene Bisphenol (25% wt.) and Dicyanate of Isopropylidene Diphenol (75% wt.)

Dicyanate of 4,4′-isopropylidene diphenol (2.5518 gram, 75% wt.) and bis(allyl ether) of cyclododecane bisphenol (0.8506 gram, 25% wt.) from Example 1 were weighed into a glass vial. HPLC analysis of the dicyanate of 4,4′-isopropylidene diphenol revealed 100 area % dicyanate. HPLC analysis of the bis(allyl ether) of 4,4′-isopropylidene diphenol revealed the presence of 99.51 area % allyl ether with the balance as 3 minor components (0.13, 0.05, and 0.31 area %). Gentle heating (did not exceed 75° C.) and mixing by swirling contents of the vial provided a solution.

DSC analysis of portions (11.40 and 12.80 milligrams) of the blend was conducted using a rate of heating of 5° C. per minute from 25° C. to 400° C. under a stream of nitrogen flowing at 35 cubic centimeters per minute. An endotherm was observed with an average 31.00° C. onset (30.29° C. and 31.71° C.), 71.48° C. minimum (71.35° C. and 71.61° C.), and 79.82° C. endpoint (78.63° C. and 81.00° C.) accompanied by an enthalpy of 64.6 joules per gram (62.10 and 67.01 joules per gram) (individual values in parenthesis). An exotherm attributed to a copolymerization of the allyl and cyanate groups (plus any homopolymerization) was observed with an average 195.70° C. onset (194.75° C. and 196.65° C.), 256.11° C. maximum (255.56° C. and 256.65° C.), and 286.94° C. endpoint (285.75° C. and 288.12° C.) accompanied by an enthalpy of 769.3 joules per gram (757.9 and 780.7 joules per gram) (individual values in parenthesis). The copolymer recovered from the DSC analysis was a transparent, amber colored, rigid solid.

| | | | Exotherm | Exotherm | |
|---|---|---|---|---|---|
| | | Exotherm | Maximum | Endpoint | Enthalpy |
| Designation | Monomer Used | Onset (° C.) | (° C.) | (° C.) | (joules/g) |
| Example 7 | 25% bis(allyl | 30.29^{a} | 74.59^{a} | 81.00^{a} | 66.59^{a} |
| | ether) of 1,1- | 196.65 | 252.51 | 289.78 | 651.8 |
| | bis(4-hydroxy- | | | | |
| | phenyl)cyclo- | | | | |
| | dodecane/ | | | | |
| | 75% dicyanate | | | | |
| | of 4,4′- | | | | |
| | isopropylidene | | | | |
| | diphenol | | | | |
| Comparative | 25% bis(allyl | 31.00^{a} | 71.48^{a} | 79.82^{a} | 64.6^{a} |
| Experiment G | ether) of 4,4′- | 195.70 | 256.11 | 286.94 | 769.3 |
| | isopropylidene | | | | |
| | diphenol/ | | | | |
| | 75% dicyanate | | | | |
| | of 4,4′- | | | | |
| | isopropylidene | | | | |
| | diphenol | | | | |

^{a}endothermic event

### Example 8

### Glass Transition Temperature of Copolymer of Bis(Allyl Ether) of 1,1-Bis(4-hydroxyphenyl)cyclododecane (25% wt.) and Dicyanate of Isopropylidene Diphenol (75% wt.)

Curing of the remaining blend from Example 7 was completed in an oven using the following curing schedule: 150° C. for 1 hour, 200° C. for 1 hour, 250° C. for 1 hour. DSC analysis of portions (31.1 and 31.8 milligrams) of the cured product gave residual exothermicity at >250° C. After a second scanning an average glass transition temperature of 176.94° C. (176.04° C. and 177.83° C.) (individual values in parenthesis) was measured with residual exothermicity followed by exothermic decomposition commencing at an average temperature of 385.04° C. (382.91° C. and 387.17° C.) (individual values in parenthesis).

### Comparative Experiment H

### Glass Transition Temperature of Copolymer of Bis(Allyl Ether) of Isopropylidene Bisphenol (25% wt.) and Dicyanate of Isopropylidene Diphenol (75% wt.)

Curing of the remaining blend from Comparative Experiment G was completed in an oven using the following curing schedule: 150° C. for 1 hour, 200° C. for 1 hour, 250° C. for 1 hour. DSC analysis of portions (30.4 and 30.8 milligrams) of the cured product gave residual exothermicity at >200° C. After a second scanning an average glass transition temperature of 162.47° C. (158.70° C. and 166.23° C.) (individual values in parenthesis) was measured with residual exothermicity followed by exothermic decomposition commencing at an average temperature of 354.2° C. (351.6° C. and 356.8° C.) (individual values in parenthesis).

| | | | Exothermic |
|---|---|---|---|
| | | | Decomposition |
| | | Tg | Onset |
| Designation | Monomer Used | (° C.) | (° C.) |
| Example 8 | 25% bis(allyl ether) of 1,1-bis(4- | 176.94 | 385.04 |
| | hydroxyphenyl)cyclododecane/ | | |
| | 75% dicyanate of 4,4′- | | |
| | isopropylidene diphenol | | |
| Comparative | 25% bis(allyl ether) of 4,4′- | 162.47 | 354.2 |
| Experiment | isopropylidene diphenol/ | | |
| H | 75% dicyanate of 4,4′- | | |
| | isopropylidene diphenol | | |

### Example 9

### Copolymerization of the Bis(Allyl Ether) of 1,1-Bis(4-hydroxyphenyl)cyclododecane (25% wt.) and 1,1-Bis(4-cyanatophenyl)cyclododecane (75% wt.) Using Catalyst

1,1-Bis(4-cyanatophenyl)cyclododecane (0.7709 gram, 75% wt.), bis(allyl ether) of cyclododecane bisphenol (0.2570 gram, 25% wt.) from Example 1, and 6% cobalt naphthenate (0.0051 gram, 0.5% wt.) were weighed into a glass vial to which dichloromethane (1.5 milliliters) was added. HPLC analysis of the 1,1-bis(4-cyanatophenyl)cyclododecane revealed 99.44 area % dicyanate and 0.56 area % monocyanate. Shaking the vial provided a solution which was added to an aluminum tray. Devolatilization conducted in a vented oven at 40° C. for 30 minutes removed the dichloromethane giving a homogeneous blend.

DSC analysis of portions (10.1 and 12.5 milligrams) of the blend was conducted using a rate of heating of 5° C. per minute from 25° C. to 400° C. under a stream of nitrogen flowing at 35 cubic centimeters per minute. An endotherm was observed with an average 51.62° C. onset (41.67° C. and 61.57° C.), 85.29° C. minimum (79.93° C. and 90.64° C.), and 93.09° C. endpoint (90.48° C. and 95.70° C.) accompanied by an enthalpy of 16.22 joules per gram (8.65 and 23.79 joules per gram) (individual values in parenthesis). An exotherm attributed to a copolymerization of the allyl and cyanate groups (plus any homopolymerization) was observed with an average 93.09° C. onset (90.48° C. and 95.70° C.), 162.04 and 238.36° C. maxima that merged together (161.28° C., 162.79° C., 236.93° C., and 239.78° C.), and 283.38° C. endpoint (282.43° C. and 284.33° C.) accompanied by an enthalpy of 422.6 joules per gram (413.0 and 432.1 joules per gram) (individual values in parenthesis). The copolymer recovered from the DSC analysis was a transparent, amber colored, rigid solid.

### Comparative Experiment I

### Copolymerization of the Bis(Allyl Ether) of Isopropylidene Diphenol (25% wt.) and Dicyanate of Isopropylidene Diphenol (75% wt.) Using Catalyst

Dicyanate of 4,4′-isopropylidene diphenol (0.7727 gram, 75% wt.), bis(allyl ether) of 4,4′-isopropylidene diphenol (0.2576 gram, 25% wt.) from Comparative Experiment A, and 6% cobalt naphthenate (0.0052 gram, 0.5% wt.) were weighed into a glass vial to which dichloromethane (1.5 milliliters) was added. HPLC analysis of the dicyanate of 4,4′-isopropylidene diphenol revealed 100 area % dicyanate. HPLC analysis of the bis(allyl ether) of 4,4′-isopropylidenediphenol revealed the presence of 99.51 area % allyl ether with the balance as 3 minor components (0.13, 0.05, and 0.31 area %). Shaking the vial provided a solution which was added to an aluminum tray. Devolatilization conducted in the vacuum oven at 40° C. for 30 minutes removed the dichloromethane giving a homogeneous blend.

DSC analysis of portions (8.7 and 11.1 milligrams) of the blend was conducted using a rate of heating of 5° C. per minute from 25° C. to 400° C. under a stream of nitrogen flowing at 35 cubic centimeters per minute. An endotherm was observed with an average 37.64° C. onset (35.50° C. and 39.77° C.), 69.39° C. minimum (69.21° C. and 69.56° C.), and 79.35° C. endpoint (79.11° C. and 79.58° C.) accompanied by an enthalpy of 50.19 joules per gram (48.64 and 51.73 joules per gram) (individual values in parenthesis). An exotherm attributed to a copolymerization of the allyl and cyanate groups (plus any homopolymerization) was observed with an average 81.48° C. onset (80.53° C. and 82.43° C.), and five maxima that merged together: 128.16° C., 166.08° C., 180.61° C., 227.93° C., and 253.76° C. (127.45° C. and 128.87° C., 165.84° C. and 166.31° C., 178.50° C. and 182.71° C., 227.45° C. and 228.40° C., 253.52° C. and 253.99° C.), and 283.85° C. endpoint (281.48° C. and 286.22° C.) accompanied by an enthalpy of 611.0 joules per gram (571.9 and 650.1 joules per gram) (individual values in parenthesis). The copolymer recovered from the DSC analysis was a transparent, amber colored, rigid solid.

| | | | Exotherm | Exotherm | |
|---|---|---|---|---|---|
| | | Exotherm | Maximum | Endpoint | Enthalpy |
| Designation | Monomer Used | Onset (° C.) | (° C.) | (° C.) | (joules/g) |
| Example 9 | 25% bis(allyl | 51.6^{a} | 85.3^{a} | 93.1^{a} | 16.2^{a} |
| | ether) of 1,1- | 93.1 | 162.0 | 283.4 | 422.6 |
| | bis(4-hydroxy- | | 238.4 | | |
| | phenyl)cyclo- | | | | |
| | dodecane/ | | | | |
| | 75% 1,1-bis(4- | | | | |
| | cyanatophenyl) | | | | |
| | cyclododecane | | | | |
| Comparative | 25% bis(allyl | 37.6^{a} | 69.4^{a} | 79.4^{a} | 50.2^{a} |
| Experiment I | ether) of 4,4′- | 81.5 | 128.2 | 283.9 | 611.0 |
| | isopropylidene | | 166.1 | | |
| | diphenol/ | | 180.6 | | |
| | 75% dicyanate | | 227.9 | | |
| | of 4,4′- | | 253.8 | | |
| | isopropylidene | | | | |
| | diphenol | | | | |

^{a}endothermic event

### Example 10

### Thermogravimetric Analysis (TGA) and Differential Scanning Calorimetry (DSC) of Copolymer of Bis(Allyl Ether) of 1,1-Bis(4-hydroxyphenyl)cyclododecane (25% wt.) and 1,1-Bis(4-cyanatophenyl)cyclododecane (75% wt.) Prepared Using Catalyst

1,1-Bis(4-cyanatophenyl)cyclododecane (3.00 grams, 75% wt.), bis(allyl ether) of 1,1-bis(4-hydroxyphenyl)cyclododecane (1.00 gram, 25% wt.) from Example 1, and 6% cobalt naphthenate (0.0040 gram, 0.1% wt.) were weighed into a glass vial to which dichloromethane (2.0 milliliters) was added. HPLC analysis of the 1,1-bis(4-cyanatophenyl)cyclododecane revealed 99.44 area % dicyanate and 0.56 area % monocyanate. Shaking the vial provided a solution which was added to a round aluminum pan. Devolatilization conducted in a vacuum oven at 50° C. for 30 minutes removed the dichloromethane giving a homogeneous blend. Curing was conducted in ovens using the following curing schedule: 100° C. for 1 hour, 150° C. for 1 hour, 200° C. for 2 hours, 250° C. for 1 hour. A rigid, transparent, amber colored disk was recovered after curing and demolding from the aluminum pan.

DSC analysis of portions (33.0 and 34.3 milligrams) of the cured product was conducted using a rate of heating of 5° C. per minute from 25° C. to 400° C. under a stream of nitrogen flowing at 35 cubic centimeters per minute. Residual exothermicity was observed at >260° C. and an average glass transition temperature of 181.83° C. (185.80° C. and 177.85° C.) (individual values in parenthesis) was measured. TGA of a portion (20.3110 milligrams) of the cured product was conducted using a rate of heating of 10° C. per minute from 25° C. to 600° C. under a dynamic nitrogen atmosphere. A step transition with an onset temperature of 400.42° C. and an end temperature of 446.57° C. was observed. The temperatures at 99.00, 95.00 and 90.00% of original sample weight were 243.23° C., 373.76° C. and 396.76° C., respectively.

### Comparative Experiment J

### Thermogravimetric Analysis (TGA) and Differential Scanning Calorimetry (DSC) of Copolymer of Bis(Allyl Ether) of Isopropylidene Diphenol (25% wt.) and Dicyanate of 4,4′-Isopropylidene Diphenol (75% wt.) Prepared Using Catalyst

Dicyanate of 4,4′-isopropylidene diphenol (3.00 grams, 75% wt.), bis(allyl ether) of 4,4′-isopropylidene diphenol (1.00 gram, 25% wt.) from Comparative Experiment A, and 6% cobalt naphthenate (0.0040 gram, 0.1% wt.) were weighed into a glass vial to which dichloromethane (2.0 milliliters) was added. HPLC analysis of the dicyanate of 4,4′-isopropylidene diphenol revealed 100 area % dicyanate. HPLC analysis of the bis(allyl ether) of 4,4′-isopropylidene diphenol revealed the presence of 99.51 area % allyl ether with the balance as 3 minor components (0.13, 0.05, and 0.31 area %). Shaking the vial provided a solution which was added to a round aluminum pan. Devolatilization conducted in a vacuum oven at 50° C. for 30 minutes removed the dichloromethane giving a homogeneous blend. Curing was conducted in ovens using the following curing schedule: 100° C. for 1 hour, 150° C. for 1 hour, 200° C. for 2 hours, 250° C. for 1 hour. A rigid, transparent, amber colored disk was recovered after curing and demolding from the aluminum pan.

DSC analysis of portions (32.3 and 34.4 milligrams) of the cured product was conducted using a rate of heating of 5° C. per minute from 25° C. to 400° C. under a stream of nitrogen flowing at 35 cubic centimeters per minute. Residual exothermicity was observed at >260° C. and an average glass transition temperature of 133.16° C. (134.03° C. and 132.29° C.) (individual values in parenthesis) was measured. TGA of a portion (6.3330 milligrams) of the cured product was conducted using a rate of heating of 10° C. per minute from 25° C. to 600° C. under a dynamic nitrogen atmosphere. A step transition with an onset temperature of 386.55° C. and an end temperature of 428.25° C. was observed. The temperatures at 99.00, 95.00 and 90.00% of original sample weight were 227.28° C., 323.19° C. and 385.32° C., respectively.

| | | | | | TGA | TGA | TGA |
|---|---|---|---|---|---|---|---|
| | | | TGA | TGA | 99.00% | 95.00% | 90.00% |
| | | | Onset | End | wt. @ | wt. @ | wt. @ |
| Designation | Monomer Used | Tg (° C.) | (° C.) | (° C.) | (° C.) | (° C.) | (° C.) |
| Example 10 | 25% bis(allyl | 181.83 | 400.42 | 446.57 | 243.23 | 373.76 | 396.76 |
| | ether) of 1,1- | | | | | | |
| | bis(4-hydroxy- | | | | | | |
| | phenyl)cyclo- | | | | | | |
| | dodecane/ | | | | | | |
| | 75% 1,1-bis(4- | | | | | | |
| | cyanatophenyl)- | | | | | | |
| | cyclododecane | | | | | | |
| Comparative | 25% bis(allyl | 133.16 | 386.55 | 428.25 | 227.28 | 323.19 | 385.32 |
| Experiment J | ether) of 4,4′- | | | | | | |
| | isopropylidene | | | | | | |
| | diphenol/ | | | | | | |
| | 75% dicyanate of | | | | | | |
| | 4,4′- | | | | | | |
| | isopropylidene | | | | | | |
| | diphenol | | | | | | |

### Example 11

### Moisture Resistance of Copolymer of Bis(Allyl Ether) of 1,1-Bis(4-hydroxyphenyl)cyclododecane (25% wt.) and 1,1-Bis(4-cyanatophenyl)cyclododecane (75% wt.) Prepared Using Catalyst

The remaining portion of the cured copolymer disk from Example 10 was weighed, added to a 4 ounce glass jar along with deionized water (40 milliliters), sealed and then placed in an oven maintained at 55° C. The disk was removed at the indicated intervals, blotted dry, weighed, and then replaced back into the sealed jar for continuation of the testing. The change in weight from the original was calculated for each time interval, providing the following results given in the table.

### Comparative Experiment K

### Moisture Resistance of Copolymer of Bis(Allyl Ether) of Isopropylidene Diphenol (25% wt.) and Dicyanate of 4,4′-Isopropylidene Diphenol (75% wt.) Prepared Using Catalyst

The remaining portion of the cured copolymer disk from Comparative Experiment J was weighed, added to a 4 ounce glass jar along with deionized water (40 milliliters), sealed and then placed in an oven maintained at 55° C. The disk was removed at the indicated intervals, blotted dry, weighed, and then replaced back into the sealed jar for continuation of the testing. The change in weight from the original was calculated for each time interval, providing the results given in the following table.

Exposure to Deionized Water at 55° C.

| Duration of | Copolymer of | Copolymer of Comparative |
|---|---|---|
| Exposure | Example 11 | Experiment K |
| (hours) | (% wt. increase) | (% wt. increase) |
| 9.0 | 0.573 | 0.700 |
| 25.33 | 0.768 | 1.016 |
| 51.83 | 0.859 | 1.222 |
| 95.16 | 1.055 | 1.400 |
| 119.08 | 1.081 | 1.469 |
| 143.00 | 1.068 | 1.510 |
| 167.17 | 1.081 | 1.537 |

### Reference Example 3

### Synthesis and Characterization of the Tetraphenol of Dimethylcyclohexane

Phenol (598 g, 6.36 moles) and cyclohexane dicarboxaldehyde (74.2 g, 0.53 moles, mixture of 1,3- and 1,4-isomers; ratio of phenolic groups to aldehyde groups=6:1, equivalent ratio of phenol to cyclohexane dicarboxaldehyde=3:1) were added together in a 1-L 5-neck reactor. The mixture was heated to 50° C. with 500 rpm mechanical stirrer agitation. At 50° C. and atmospheric pressure, p-toluenesulfonic acid (PTSA) (1.3959 g total, 0.207% by weight) was added in six portions over 30 minutes. The temperature increased a few degrees with each PTSA addition. After the 6th PTSA addition, the temperature controller was set to 70° C. and vacuum was applied to the reactor. In order to avoid the reactor content flooding the rectifier, the reactor pressure was gradually decreased to remove water from the reaction solution. When the reflux had stopped, the reactor was vented and water (48 g) was added.

Water (79 g) and NaHCO₃ (0.6212 g) were added to neutralize the PTSA. When the reaction contents had cooled to room temperature, the entire contents were transferred to a 2-L separatory funnel. Methyl ethyl ketone (MEK) was added, and the contents were washed several times with water to remove PTSA-salt. The solvents and excess phenol were removed using a rotary evaporator, and the hot novolac was poured onto aluminum foil. The reaction of phenol with cyclohexane dicarboxaldehyde produced as the predominant product a tetraphenol possessing the following idealized structure (tetraphenol of dimethylcyclohexane):

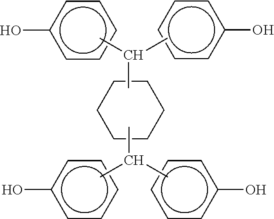

Ultraviolet spectrophotometric analysis provided a hydroxyl equivalent weight (HEW) of 118.64. High pressure liquid chromatographic (HPLC) analysis was adjusted to resolve 24 (isomeric) components present in the product.

Although the present invention has been described in considerable detail with regard to certain versions thereof, other versions are possible, and alterations, permutations, and equivalents of the version shown will become apparent to those skilled in the art upon a reading of the specification and study of the drawings. Also, the various features of the versions herein can be combined in various ways to provide additional versions of the present invention. Furthermore, certain terminology has been used for the purposes of descriptive clarity, and not to limit the present invention. Therefore, any appended claims should not be limited to the description of the preferred versions contained herein and should include all such alterations, permutations, and equivalents as fall within the true spirit and scope of the present invention.

Having now fully described this invention, it will be understood to those of ordinary skill in the art that the methods of the present invention can be carried out with a wide and equivalent range of conditions, formulations, and other parameters without departing from the scope of the invention or any embodiments thereof.

1. An ethylenically unsaturated monomer of formula (I): 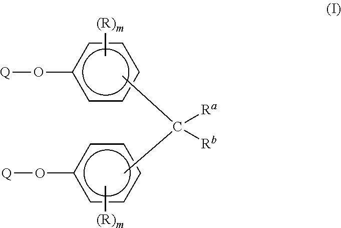
wherein:
each m independently is 0, 1, or 2;
the moieties R^{a} and R^{b} independently represent optionally substituted aliphatic groups comprising a total of from about 5 to about 24 carbon atoms, and R^{a} and R^{b} together with the carbon atom to which they are bonded may form an optionally substituted and/or optionally unsaturated and/or optionally polycyclic aliphatic ring structure; and
the moieties R independently represent halogen, cyano, nitro, hydroxy, amino optionally carrying one or two alkyl groups, optionally substituted alkyl, optionally substituted cycloalkyl, optionally substituted alkoxy, optionally substituted alkenyl, optionally substituted alkenyloxy, optionally substituted aryl, optionally substituted aralkyl, optionally substituted aryloxy, and optionally substituted aralkoxy; and
the moieties Q independently represent hydrogen, HR¹C═CR¹—CH₂— or H₂R¹C—CR¹═HC—, wherein the moieties R¹ independently represent hydrogen or optionally substituted alkyl having from 1 to about 3 carbon atoms;
with the proviso that when both moieties Q are hydrogen and R^{a} and R^{b} together with the carbon atom to which they are bonded do not form an aliphatic ring structure comprising at least about 8 ring members, at least one moiety R represents HR¹C═CR¹—CH₂— or H₂R¹C—CR¹═HC—.

2. The ethylenically unsaturated monomer of claim 1, wherein the monomer is of formula (Ia): 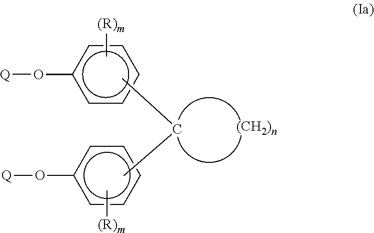
wherein:
m, R and Q are as defined in claim 1; and
n has a value of from about 5 to about 24;
with the proviso that when both moieties Q are hydrogen, at least one moiety R represents HR¹C═CR¹—CH₂— or H₂R¹C—CR¹═HC—;
and any non-aromatic cyclic moieties comprised in the above formula (Ia) may optionally carry one or more substituents and/or may optionally comprise one or more double bonds and/or may optionally be polycyclic.

3. The monomer of claim 2, wherein n has a value of from about 9 to about 16.

4. The monomer of claim 2, wherein n has a value of 9, 10, or 11.

5. The monomer of claim 1, wherein each m independently is 0 or 1.

6. The monomer of claim 1, wherein the moieties Q independently represent HR¹C═CR¹—CH₂— or H₂R¹C—CR¹═HC—.

7. The monomer of claim 1, wherein the moieties R¹ independently represent hydrogen or methyl.

8. The monomer of claim 1, wherein the moieties Q are identical and represent allyl, methallyl, or 1-propenyl.

9. The monomer of claim 1, which is 1,1-bis(4-hydroxyphenyl)cyclododecane bis(allyl ether).

10. An ethylenically unsaturated monomer of formula (II): 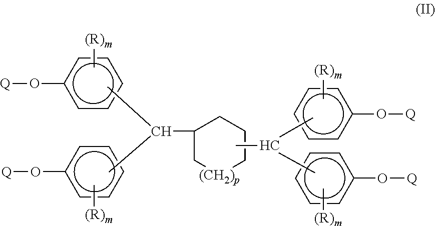
wherein:
p is 0 or an integer of from 1 to about 19;
each m independently is 0, 1, or 2;
the moieties R independently represent halogen, cyano, nitro, hydroxy, amino optionally carrying one or two alkyl groups, optionally substituted alkyl, optionally substituted cycloalkyl, optionally substituted alkoxy, optionally substituted alkenyl, optionally substituted alkenyloxy, optionally substituted aryl, optionally substituted aralkyl, optionally substituted aryloxy, and optionally substituted aralkoxy; and
the moieties Q independently represent hydrogen, HR¹C═CR¹—CH₂— or H₂R¹C—CR¹═HC—, wherein the moieties R¹ independently represent hydrogen or optionally substituted alkyl having from 1 to about 3 carbon atoms, with the proviso that when all four moieties Q are hydrogen, at least one moiety R represents HR¹C═CR¹—CH₂— or H₂R¹C—CR¹═HC—;
and any non-aromatic cyclic moieties comprised in the above formula (II) may optionally carry one or more substituents and/or may optionally comprise one or more double bonds.

11. The monomer of claim 10, wherein p has a value of from 1 to about 14.

12. The monomer of claim 10, wherein p has a value of 1, 2, or 3.

13. The monomer of claim 10, wherein each m independently is 0 or 1.

14. The monomer of claim 10, wherein the moieties Q independently represent HR¹C═CR¹—CH₂— or H₂R¹C—CR¹═HC—.

15. The monomer of claim 10, wherein the moieties R¹ independently represent hydrogen or methyl.

16. The monomer of claim 10, wherein the moieties Q are identical and represent allyl, methallyl, or 1-propenyl.

17. The monomer of claim 10, which is dimethylcyclohexane tetraphenol tetra(allyl ether).

18. A polymer or prepolymer of a monomer of claim 1.

19. A polymerizable mixture, wherein the mixture comprises at least two of (i) at least one monomer of claim 1 and/or a prepolymer thereof, (ii) at least one monomer of formula II and/or a prepolymer thereof, and (iii) at least one monomer and/or a prepolymer thereof which is different from (i) and (ii).

20. The mixture of claim 19, wherein the at least one monomer (iii) is selected from monomers which comprise one or more polymerizable ethylenically unsaturated moieties, aromatic di- and polycyanates, aromatic di- and polycyanamides, di- and polymaleimides, and di- and polyglycidyl ethers.

21. The mixture of claim 19, wherein the mixture comprises at least (i) and (iii).

22. The mixture of claim 19, wherein the mixture comprises at least (ii) and (iii).

23. The mixture of claim 19, wherein (iii) comprises a dicyanate compound of formula (III) and/or a prepolymer thereof: 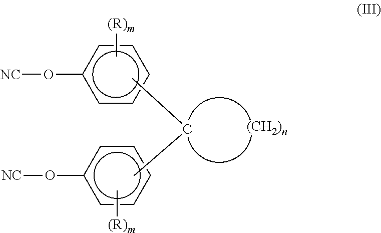
wherein:
n has a value of from about 5 to about 24;
each m independently is 0, 1, or 2; and
the moieties R independently represent halogen, cyano, nitro, optionally substituted alkyl, optionally substituted cycloalkyl, optionally substituted alkoxy, optionally substituted alkenyl, optionally substituted alkenyloxy, optionally substituted aryl, optionally substituted aralkyl, optionally substituted aryloxy, and optionally substituted aralkoxy;
and any non-aromatic cyclic moieties comprised in the above formula (III) may optionally carry one or more substituents and/or may optionally comprise one or more double bonds.

24. The mixture of claim 23, wherein the dicyanate compound of formula (III) comprises 1,1-bis(4-cyanatophenyl)cyclododecane.

25. The mixture of claim 19, wherein (iii) comprises a polycyanate compound of formula (IV) and/or a prepolymer thereof: 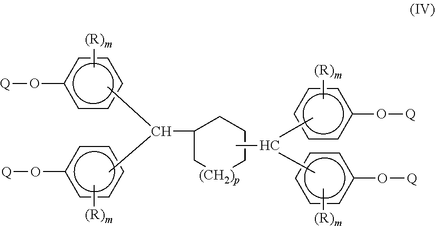
wherein:
p is 0 or an integer of from 1 to about 19;
each m independently is 0, 1, or 2;
the moieties R independently represent halogen, cyano, nitro, optionally substituted alkyl, optionally substituted cycloalkyl, optionally substituted alkoxy, optionally substituted alkenyl, optionally substituted alkenyloxy, optionally substituted aryl, optionally substituted aryloxy, and optionally substituted aralkoxy; and
at least two of the moieties Q represent —CN and the remaining moieties Q represent hydrogen;
and any non-aromatic cyclic moieties comprised in the above formula (IV) may optionally carry one or more substituents and/or may optionally comprise one or more double bonds.

26. The mixture of claim 25, wherein in formula (IV) all four moieties Q represent —CN.

27. The mixture of claim 26, wherein the polycyanate compound of formula (IV) comprises dimethylcyclohexane tetraphenol tetracyanate.

28. The mixture of claim 19, wherein the mixture further comprises one or more substances which are selected from polymerization catalysts, co-curing agents, flame retardants, synergists for flame retardants, solvents, fillers, adhesion promoters, wetting aids, dispersing aids, surface modifiers, thermoplastic polymers, and mold release agents.

29. A mixture comprising at least one monomer of claim 1 and/or a prepolymer thereof and one or more substances which are selected from polymerization catalysts, co-curing agents, flame retardants, synergists for flame retardants, solvents, fillers, adhesion promoters, wetting aids, dispersing aids, surface modifiers, thermoplastic polymers, and mold release agents.

30. The mixture of claim 29, wherein the mixture is partially or completely polymerized.

31. A product which comprises a polymerized mixture of claim 19.

32. The product of claim 31, wherein the product is at least one of an electrical laminate, an IC substrate, a casting, a coating, a die attach and mold compound formulation, a composite, and an adhesive.

33. A process for preparing a mixture of ethylenically unsaturated monomers, wherein the process comprises condensing a dialdehyde of a cycloalkane having from about 5 to about 24 ring carbon atoms with a hydroxyaromatic compound at a ratio of aromatic hydroxy groups to aldehyde groups which results in a mixture of polyphenolic compounds with a polydispersity of not higher than about 2 and subjecting the mixture of polyphenolic compounds to an etherification reaction to partially or completely convert phenolic groups present in the mixture into groups of formula HR¹C═CR¹—CH₂—O— and/or H₂R¹C—CR¹═HC—O— wherein the moieties R¹ independently represent hydrogen or unsubstituted or substituted alkyl having from 1 to about 3 carbon atoms.

34. The process of claim 33, wherein the ratio of aromatic hydroxy groups to aldehyde groups is at least about 4.

35. The process of claim 33, wherein the cycloalkane has 6, 7 or 8 carbon atoms.

36. The process of claim 33, wherein the dialdehyde comprises cyclohexane dicarboxaldehyde and the hydroxyaromatic compound comprises phenol.

37. A mixture of ethylenically unsaturated monomers which is obtainable by the process of claim 33.
